(19) United States
(12) Patent Application Publication
(10) Pub. No.: US 2024/0100183 A1
(43) Pub. Date: Mar. 28, 2024

(54) COMPOSITIONS COMPRISING MOLECULES FOR CYSTIC FIBROSIS TREATMENT

(71) Applicant: UNiversity of Iowa Research Foundation, Iowa City, IA (US)

(72) Inventors: Aliasger K. Salem, Iowa City, IA (US); David A. Stoltz, Iowa City, IA (US); Michael J. Welsh, Riverside, IA (US); Sarah Ernst, Kalona, IA (US); Youssef W. Naguib, Minia (EG); David S. NAkhla, Iowa City, IA (US); Aml I. Mekkawy, Sohag (EG); Nikesh Gupta, Iowa City, IA (US)

(21) Appl. No.: 18/256,593

(22) PCT Filed: Dec. 10, 2021
(86) PCT No.: PCT/US2021/062913

§ 371 Date: Jun. 8, 2023

Related U.S. Application Data
(60) Provisional application No. 63/124,388, filed on Dec. 11, 2020.
(60) Provisional application No. 63/232,455, filed on Aug. 12, 2021.

(51) Int. Cl. A61K 47/69; A61K 9/00; A61K 9/14; A61K 31/47; A61K 45/06

(52) U.S. Cl. CPC A61K 47/6951; A61K 9/0019; A61K 9/14; A61K 31/47; A61K 45/06

(57) ABSTRACT

Composition and methods to prevent, inhibit or treat one or more symptoms of cystic fibrosis are provided.

### CROSS-REFERENCE TO RELATED APPLICATIONS

This application claims the benefit of the filing date of U.S. application No. 63/124,388, filed on Dec. 11, 2020, and U.S. application No. 63/232,455, filed on Aug. 12, 2021, the disclosures of which are incorporated by reference herein.

### STATEMENT OF GOVERNMENT RIGHTS

This invention was made with government support under NIH P30 CA086862 awarded by the National Institutes of Health. The government has certain rights in the invention.

### BACKGROUND

Cystic fibrosis (CF) is an autosomal recessive genetic disorder caused by mutation in cystic fibrosis transmembrane conductance regulator (CFTR) gene (located at chromosome 7q31.2), which encodes for CFTR chloride channel protein responsible for maintaining water and ion balance intra- and extracellularly (Ipka et al., 2014). This serious life-shortening condition affects 30,000 patients in the United States of America and 70,000 worldwide. CF is caused by a defect in CFTR function which blocks chloride and bicarbonate ion flow across epithelial cells resulting in mucus build-up at the apical surface of epithelial cells, and deterioration of the functions of major organs such as pulmonary, gastrointestinal, hepatic, endocrine and reproductive systems consequently follows, which ultimately ends up with organ failure (Condren et al., 2013). Increased susceptibility to various bacterial lung infections, especially Pseudomonas aeruginosa, is common in CF patients, and markedly participate in the general health deterioration and respiratory failure, which is the major cause of death in CF patients. Five classes of CF mutations have been identified

Currently, there are approximately over 1900 disease-causing CFTR reported mutations. These mutations are grouped into five classes based on the type of mutation and its resulting phenotype. Class I mutation involves truncated protein translation, while class II mutations cause misfolded CFTR. These two defects result in the inability of the CFTR protein to translocate to the cell surface. The third and fourth classes' defects result in a fully translated CFTR able to translocate to cell surface, but it exhibits gating dysfunction or decreased pore conductivity. The last class, class five defects, causes a reduction in CFTR protein expression at the cell surface (Condren et al., 2013)

Ivacaftor (VX-770, N-(2,4-Di-tert-butyl-5-hydroxyphenyl)-4-oxo-1,4-dihydroquinoline-3-carboxamide; IVA; FIG. 1) is a selective small molecule CFTR modulator that targets the 3rd most common genetic mutation in the CFTR gene (G551D—Class III—gating mutation, occurring in 5-6% of CF patients) (Clancy et al., 2014; Fohner et al., 2017). It restores the defective CFTR function by increasing the opening probability of CFTR channels at the cell surface (CFTR gate ‘potentiator’). It is the first CFTR modulator to be approved in 2012 after being developed by Vertex® pharmaceuticals in an amorphous solid dispersion tablets form under the trade name Kalydeco® (Fohner et al., 2017, Deeks, 2016). It is administered orally at a dose of 150 mg twice daily owing to its relatively short half-life of only 9-12 h (Deeks, 2016). IVA has low water solubility (<0.05 μg/mL) and is very lipophilic (log p=3.3), which is why, when Kalydeco® is administered orally, it is recommended with a fatty meal to aid in its stomach absorption, improve its bioavailability and overall exposure. It has been reported that adults with CF are more likely to be non-compliant to CF treatment, due to the chronic nature of CF and the need for multiple daily medications (Conway et al., 1996).

### SUMMARY

In one embodiment, the disclosure provides for a composition comprising a carrier comprising, in one embodiment, a cyclodextrin or a surfactant, e.g., water soluble, and one or more cystic fibrosis transmembrane conductance regulator (CFTR) modulators. In one embodiment, the compositions provide for increased solubility of the modulator(s) and/or increased bioavailability. In one embodiment, the surfactant is a cationic surfactant. In one embodiment, the surfactant is an anionic surfactant. In one embodiment, the surfactant is a zwitterionic surfactant. In one embodiment, the surfactant is a non-ionic surfactant. In one embodiment, the CFTR modulator is lipophilic. In one embodiment, at least one of the CFTR modulators is a CFTR potentiator. In one embodiment, the CFTR modulator comprises ivacaftor, GLPG2451, GLPG1837, QBW251, PTI-808, FDL176, FDL169, quercetin, genistein, rattlesnake phospholipase A2 or aminoarylthiazone. In one embodiment, at least one of the CFTR modulators is a CFTR corrector. In one embodiment, the CFTR corrector comprises lumacaftor, Corr-4a, VRT-325, tezacaftor, elexacaftor, cavosonstat, FDL169, VX-152, VX-440, VX-445, or VX-659. In one embodiment, the composition comprises a CFTR potentiator and a CFTR corrector. In one embodiment, the composition comprises ivacaftor and lumacaftor, tezacaftor, or elexacaftor. In one embodiment, the cyclodextrin is an unmodified cyclodextrin. In one embodiment, the cyclodextrin is a modified cyclodextrin. In one embodiment the modified cyclodextrin is modified with hydroxymethyl, hydroxyethyl, hydroxypropyl, or hydroxybutyl. In one embodiment, the carrier comprises one or a binary or a ternary mixture of α-, β, γ, HP-β-CD, M-β-CD, or HP-γ-CD. In one embodiment the modified cyclodextrin is modified with hydroxymethyl, hydroethyl, hydroxypropyl, hydroxybutyl, dihydroxypropyl, hydroxybutyl, methyl, ethyl, butyl, acetyl, succinyl, glucosyl, maltosyl, sulfobutyl-ether, per-C-acetyl, per-O-butanoyl, per-O-valeryl, per-O-octyl, carboxymethyl, or O-carboxymethyl-O-ethyl. In one embodiment, the surfactant is a polyol. In one embodiment, the surfactant is a glycol, glycerol ester or a sorbitan or ethoxylated modified polyol. In one embodiment, the composition is a tablet. In one embodiment, the composition is a suspension of a powder in a pharmaceutically acceptable oral vehicle. In one embodiment, the molar ratio between the CFTR modulator and the carrier is 3:1 to 1:10. In one embodiment, the molar ratio between the CFTR modulator and the carrier is 4:1 to 1:8. In one embodiment, the molar ratio between the CFTR modulator and the carrier is 2:1 to 1:20. In one embodiment, the molar ratio between the CFTR modulator and the carrier is 2:1 to 1:8. In one embodiment, the molar ratio between the CFTR modulator and the carrier is 2:1 to 1:5. In one embodiment, the molar ratio between the CFTR modulator and the carrier is 4:1 to 1:20. In one embodiment, the carrier comprises polysorbate 80. In one embodiment, the amount of surfactant in the composition is 1 to 25% v/v, e.g., for injection. In one embodiment the amount of surfactant in the composition is 5% to 10% or 12.5% to 25% v/v of, for example, polysorbate 80 (polyoxyethylene (20) sorbitan monooleate). In one embodiment, the amount of surfactant in the composition is 50 to 90% v/v, e.g., for oral administration, e.g., 50 to 70% v/v of, for example, polysorbate 80. In one embodiment, the surfactant comprises polysorbate 20 (Tween 20; polyethylene(20) sorbitan monolaurate), polysorbate 80 (Tween 80; polyethylene(20) sorbitan monooleate), a lower alkyl, e.g., C1-C6, alcohol, e.g., ethanol, a glycol or polyol, e.g., polyethylene glycol, propylene glycol, or a mixture of thereof.

Also provided is a method to prepare a composition comprising a cyclodextrin and one or more cystic fibrosis transmembrane receptor (CFTR) modulators. The method includes mixing one or more CFTR modulators and a cyclodextrin in a solution comprising water and an alcohol at ratios of 1:5 to 5:1. e.g., 2:1 to 1:2; and drying the resulting mixture. In one embodiment, the mixing is accomplished using a mortar and pestle. In one embodiment, a vacuum is employed for drying. In one embodiment, the mixing is accomplished by dissolving the one or more CFTR modulators in a compatible solvent and combining it with an aqueous solution of the cyclodextrin.

Further provided is a method to inhibit or treat one or more symptoms, or to treat the CFTR defect, of cystic fibrosis in a mammal. The method includes administering to a mammal in need thereof a composition comprising one or more carriers comprising a cyclodextrin or a surfactant and an effective amount of one or more CFTR modulators. In one embodiment, the mammal is a human. In one embodiment, the composition is orally administered. In one embodiment, the composition is intramuscularly administered. In one embodiment, the composition is subcutaneously administered. In one embodiment, the composition is intravenously administered. In one embodiment, at least one of the CFTR modulators is a CFTR potentiator. In one embodiment, the CFTR modulator comprises ivacaftor, GLPG2451, GLPG1837, QBW251, PTI-808, FDL176, FDL169, quercetin, genistein, rattlesnake phospholipase A2 or aminoarylthiazone. In one embodiment, at least one of the CFTR modulators is a CFTR corrector. In one embodiment, the CFTR corrector comprises lumacaftor, Corr-4a. VRT-325, tezacaftor, elexacaftor, cavosonstat, FDL169. VX-152, VX-440, VX-445, or VX-659. In one embodiment, the composition comprises a CFTR potentiator and a CFTR corrector. In one embodiment, a CFTR potentiator, e.g., in a composition having a carrier, is administered separately from a CFTR corrector, e.g., in a composition having a carrier. In one embodiment, the composition comprises ivacaftor and lumacaftor, tezacaftor, or elexacaftor, or salt thereof. In one embodiment, the CFTR potentiator or CFTR corrector is a salt of ivacaftor, lumacaftor, tezacaftor, or elexacitor, e.g., ivacaftor, lumacaftor, tezacaftor, or elexacftor hydrochloride, sodium ivacaftor, lumacaftor, tezacaftor, or elexacftor, ivacaftor, lumacaftor, tezacaftor, or elexacftor sulfate, ivacaftor, lumacaftor, tezacaftor, or elexacftor acetate, ivacaftor, lumacaftor, tezacaftor, or elexacftor phosphate, ivacaftor, lumacaftor, tezacaftor, or elexaciftor diphosphate, ivacaftor, lumacaftor, tezacaftor, or elexacftor chloride, potassium ivacaftor, lumacaftor, tezacaftor, or elexacftor, ivacaftor, lumacaftor, tezacaftor, or elexacftor maleate, calcium ivacaftor, lumacaftor, tezacaftor, or elexaciftor, ivacaftor, lumacaftor, tezacaftor, or elexaciftor citrate, ivacaftor, lumacaftor, tezacaftor, or elexaciftor mesylate, ivacaftor, lumacaflor, tezacaftor, or elexacftor nitrate, ivacaftor, lumacaftor, tezacaftor, or elexacftor tartrate, aluminum ivacaftor, lumacaftor, tezacaftor, or elexaciftor, or ivacaftor, lumacaftor, tezacaftor, or elexaciftor gluconate. In one embodiment, the cyclodextrin is a modified cyclodextrin. In one embodiment, the surfactant is a non-ionic surfactant, e.g., a glycol, glycerol ester or a sorbitan or ethoxylated modified polyol. In one embodiment, the molar ratio between the CFTR modulator and the carrier is 3:1 to 1:10. In one embodiment, the amount of surfactant in the composition is 1 to 25% v/v. In one embodiment, the surfactant comprises polysorbate 20, polysorbate 80, ethanol, polyethylene glycol, propylene glycol, or a mixture of thereof. In one embodiment, the mammal is a human with a class I or class II defect. In one embodiment, the mammal is a human with a class III or class IV defect.

In one embodiment, compositions comprising a formulation comprising a CFTR potentiator, e.g., ivacaftor, optionally in combination or conjunction with other CFTR modulators, e.g., ones that improve the pharmacokinetics and efficacy of the potentiator, are envisioned. In one embodiment, the formulation comprises ivacaftor and a cyclodextrin which may provide for improved in vitro dissolution, oral pharmacokinetics, and/or efficacy against manifestations of cystic fibrosis, especially in utero complications.

In one embodiment, the composition comprises a formulation having a CTFR potentiator, e.g., a water-soluble form of ivacaftor (VX-770) or other CFTR modulator, and a non-ionic surfactant such as a polysorbate, e.g., polysorbate 80 (Tween 80®, polyoxyethylene sorbitan monooleate), which may be suitable for, for example, intravenous (IV), intramuscular (IM), or subcutaneous (SC) injection, in addition to oral administration. The amount of the non-ionic surfactant, e.g., Polysorbate 80, may range from 1 to 25% v/v of the aqueous-based solution. Other solubilizers may also be utilized, either alone or in combination with polysorbate 80 to achieve solubilization in aqueous solutions.

To provide for sustained release formulations of CFTR gene modulators, in one embodiment, injectable particles, e.g., microparticles or pellets, were prepared using the biodegradable polymer poly(D,L-lactide-co-glycolide) (PLGA) and loaded them with ivacaftor. Ivacaftor has not been encapsulated in an injectable long-acting particle or pellet formulation before. These injectable particles are intended to provide a sustained release of ivacaftor to eliminate the need for its frequent daily administration. To develop the particles, in one embodiment, a simple oil-in-water emulsion technique was used both the ivacaftor and PLGA were dissolved in an organic (oil) solvent then added that solution to water with some surfactant (soap like chemical) to help the organic solution mix with water. This mixture was then stirred, followed by evaporation of the organic solvent, then collection and washing of the resulting microparticles. A total of 6 particle formulations were prepared and their characteristics assessed. One formulation, with the desirable characteristics, was selected and injected sub-cutaneously (SC) into mice. This formulation successfully provided sustained plasma (blood) levels of ivacaftor for up to 28 days following its SC injection in mice. These results indicate that the ivacaftor-loaded particle formulation could provide steady plasma levels of ivacaftor for up to a month in mice following a single injection. This injectable formulation may eliminate the need of multiple daily administrations of ivacaftor and thus improve patient's lifestyle, compliance to treatment, and treatment outcomes. Particle formulations may be employed for subcutaneous (SC) injection, thereby providing for a sustained release of the CFTR modulator loaded microparticle. In one embodiment, a hot melt extrusion method was employed to produce the pellets. In one embodiment, the pellets have a diameter of about 0.5 mm to about 1.3 mm, e.g., 0.5 mm to about 1 mm or about 0.6 mm to about 0.8 mm.

The disclosure thus provides for a sustained release formulation comprising a plurality of particles, e.g., microparticles or pellets, comprising one or more CFTR modulators. In one embodiment, the sustained release formulation provides for release over at least 2 to 8 weeks, e.g., about 4 to 6 weeks. In one embodiment, the formulation is injectable. In one embodiment, the sustained release formulation comprises particles formed of a synthetic polymer, such as a poly(lactic-co-glycolic) (PLGA) copolymer, e.g., the polymer in a particle is from about 70% w/w to about 95% w/w, e.g., from about 85% w/w to about 95% w/w, e.g., about 90% w/w. In one embodiment, the synthetic polymer comprises PLGA with a lactic acid to glycolic acid ratio of from about 60:40 to about 40:60, e.g., about 55:45 or 45:55, e.g., 50:50 The one or more compounds including at least one CFTR modulator is/are present in a particle (e.g., relative to the total amount of polymer) at about 1% w/w to about 20% w/w, e.g., about 5% w/w to about 15% w/w, including about 10% w/w.

In one embodiment, the sustained release formulation comprises particles formed of a synthetic polymer comprising 80% w/w to less than 100% w/w of a PLGA copolymer with a ratio of lactic and glycolic acids between 0:100 and 100:0, e.g., about 50:50, 45:55, 40:60, 60:40 or 55:45, and from 0.01 w/w to 20% w/w of an active pharmaceutical ingredient, as disclosed herein. In one embodiment, a composition having a plurality of different particles, such as particles having different ratios of lactic acid to glycolic acid, or a plurality of particles having different diameters, for example, microparticles having a diameter of about 5 to about 10 microns, about 4 to about 8 microns, about 8 to about 15 microns or more, are envisioned.

The materials used to manufacture the particle (polymer, lipid, or any other materials mentioned herein) can be used alone or in combination with a release modifier (e.g., isopropyl myristate, polymer blend, amphiphilic materials like Labrasol®, Labrafac®, lecithin or lecithin derivatives, Tween, Span, Brij, Pluronics, or similar materials), a porosity enhancing materials (porogens) like different grades of polyethylene glycol, or coated with a proper coating material that may form a microcapsule structure like Eudragits® or other methacrylate derivatives, shellac or other waxes, or any other material known in the art to coat the microparticles.

In one embodiment, one or more CFTR modulators are encapsulated in poly(lactide-co-glycolide) (PLGA) polymer in the form of spherical particles in the micron size (e.g., from about 5 to about 50 μm) or pellets having a diameter of, for example 0.6 mm to 8 mm. PLGA is a biodegradable and biocompatible polymer that degrades into safe, non-toxic, non-inflammatory material when injected in the body. The degradation rate of the polymer is dependent on its chemistry. e.g., the ratio of lactic to glycolic acid monomers, and other factors such as the particle size.

In one embodiment, the particles may comprise a CFTR modulator that alters read through, e.g., ELX-02 or Ataluren (PTC124), is a CFTR corrector, e.g., Lumacaftor (VX-809), Tezacaftor (VX-661), Elexacaftor (VX-445), Bamocaftor (VX-859), Olacaftor (VX-440), Posenacaftor (PTI-801), Galicaftor (ABBV-2222), ABBV-3221, or FDL169, a CFTR stabilizer such as Cavosonstat (N91115), a CFTR potentiator such as Ivacaftor (VX-770), Deutivacaftor (VX-561) or ABBV-974 (GLPG-1837), or a CFTR amplifier, e.g., Nesolicator (PTI-428). The microparticles may include more than one CFTR modulator, e.g., a combination of elexacaftor/tezacaftor/ivacaftor, lumacaftor/ivacaftor or tezacaftor/ivacaftor.

In one embodiment, particles formed of a synthetic polymer, e.g., PLGA, comprise a lipophilic compound, e.g., logp=3.3, having low water solubility (<0.05 μg/mL). In one embodiment, the compound comprises lumacaftor, tezacaftor, elexacaftor, olacaftor, selective estrogen receptor modulators (SERM), selective androgen receptor modulators (SARM), anti-pregnancy medications, leuprolide acetate or etonogestrel.

The disclosure also provides for a method to prevent, inhibit or treat one or more symptoms of cystic fibrosis in a mammal, comprising administering to the mammal an effective amount of a composition comprising one or more CFTR modulators. In one embodiment, the mammal is a human. In one embodiment, the composition is injected. In one embodiment, the composition is locally administered. In one embodiment, the composition is systemically administered. In one embodiment, the composition comprises microparticles formed of a polymer having a Mw of about 24,000 to about 38,000. In one embodiment, the one or more CFTR modulators are released over 1 to 12 weeks, e.g., released for at least 2 weeks, 4 weeks, 6 weeks, 8 weeks or 10 weeks.

The particles or pellets may be used to prevent, inhibit or treat diseases other than cystic fibrosis, diseases which are benefited by administration of CFTR modulators.

### BRIEF DESCRIPTION OF THE FIGURES

FIG. 1. Left: phase solubility of ivacaftor in different concentrations of α, β, γ, and HPβCDs (n=3, values are means t SD). Right: phase solubility of ivacaftor in α, β, and γCDs at a smaller scale of Y-axis to show differences (n=3, values are means±SD).

FIG. 2. DSC thermograms of ivacaftor, HPβCD, physical mixture, and ivacaftor/HPβCD complex (molar ratio 1:2).

FIG. 3. FTIR spectra of ivacaftor, HPβCD, and ivacaftor/HPβCD complex (molar ratio 1:2).

FIGS. 4A-4D. Powder X-ray diffraction patterns of A) ivacaftor/HPβCD complex (molar ratio 1:2), B) physical mixture, C) HPβCD, and D) ivacaftor

FIG. 5A-5D. SEM photomicrographs of a. ivacaftor, b. HPβCD, c. physical mixture, d. ivacaftor/HPβCD complex (molar ratio 1:2)

FIG. 6. Dissolution profiles of ivacaftor, ivacaftor/HPβCD complex (molar ratio 1:2), and Kalydeco® (n=1).

FIG. 7. Pharmacokinetic profile following IV injection of 0.1 mg/mouse (n=3) in female Balb-c mice (left). Pharmacokinetic parameters following 2-compartment model fitting (right).

FIG. 8. Pharmacokinetic profiles following PO (per oral) administration of 20 mg/kg Kalydeco® or ivacaftor/HPβCD complex (molar ratio 1:2) to Balb-c mice (approximately 25 g) followed by fitting the data to 2-compartmental model using PK Solver Excel add-in. Above: Fitting of the data to 2-compartmental analysis. Below: Pharmacokinetics parameters following 2-compartmental model analysis.

FIG. 9. Plasma levels in piglets (approximately 10 kg each) following the administration of 20 mg/kg of ivacaftor solution (in 20% v/v Tween 80) by intramuscular injection and ivacaftor/HPβCD complex (molar ratio 1:2) orally.

FIG. 10. Plasma levels in pregnant sow (151 kg) following PO administration of 4 doses of 20 mg/kg ivacaftor/HPβCD complex (molar ratio 1:2) at 0, 12, 24, and 36 h (n=1). Black diamond represents fetal liver ivacaftor levels (n=3, 3.0 t 0.20).

FIG. 11. Plasma levels in pregnant sow (124 kg) following IM injection of 20 mg/kg ivacaftor solution (in 20% v/v Tween 80) as 6 injections of 20 ml each (n=1). Black triangle represents fetal liver ivacaftor levels (n=3, 9.1*0.83).

FIG. 12. Plasma levels in pregnant sow (145 kg) following bid oral doses of 5 mg/kg ivacaftor/HPβCD complex (molar ratio 1:2) starting on the 35^{th} until the 120^{th} gestational day (n=1).

FIGS. 13A-13D. Ivacaftor (bid 5 mg/kg ivacaftor/HPβCD complex (molar ratio 1:2)) administration to pregnant sow alleviates meconium ileus in G551D/G551D CF piglets in utero. A) Wild-type newborn pig intestinal tract. Black arrow denotes normal-appearing spiral colon. B) G551D/G551D-CFTR newborn pig intestinal tract. Black arrow denotes microcolon. C) Null-CFTR newborn pig intestinal tract. Black arrow denotes microcolon. D) Intestinal tract from a G551D/G551D-CFTR newborn pig treated in utero with Ivacaftor (bid 5 mg/kg ivacaflor/HPβCD complex (molar ratio 1:2)). Black arrow denotes relatively normal-appearing and -sized spiral colon.

FIGS. 14A-14D. Ivacaftor (bid 5 mg/kg ivacaftor/HPβCD complex (molar ratio 1:2)) administration to pregnant sow reduces pancreatic destruction in G551D/G551D CF piglets in utero. A) Wild-type newborn pig pancreas. B) G551D/G551D-CFTR newborn pig pancreas. C) Null-CFTR newborn pig pancreas. D) Pancreas from a G551D/G551D-CFTR newborn pig treated in utero with Ivacaftor (bid 5 mg/kg ivacaftor/HPβCD complex (molar ratio 1:2)).

FIGS. 15A-15D. Ivacaftor (bid 5 mg/kg ivacaftor/HPβCD complex (molar ratio 1:2)) administration to pregnant sow corrects micro-gallbladder in G551D/G551D CF piglets. A) Wild-type newborn pig gallbladder. B) G551D/G551D-CFTR newborn pig micro-gallbladder. C) Null-CFTR newborn pig micro-gallbladder. D) Gallbladder from G551D/G551D-CFTR newborn pig treated in utero with Ivacaftor (bid 5 mg/kg ivacaftor/HPβCD complex (molar ratio 1:2).

FIG. 16. Structure of ivacaftor.

FIG. 17. Structure of poly(D,L-lactide-co-glycolide) (PLGA).

FIG. 18. Bar plot showing the average particle sizes of formulations 1 through 6. Error bars represent the standard deviation (SD). N=100 particles were measured from each formulation from scanning electron microscopy images using ImageJ® software.

FIG. 19. Particle size distribution histograms of formulations 1 through 6 showing unimodal gaussian distribution indicating homogenous particle size distribution

FIG. 20. Scanning electron photomicrographs of the ivacaftor-loaded microspheres of formulations 1 through 6.

FIGS. 21A-21D. In vitro cumulative release profiles of ivacaftor-loaded formulations 1 through 6 showing the effect of (A) drug loading. (B) PVA surfactant concentration, (C) stirring speed, (D) PLGA molecular weight. Error bars represent mean±SE (n=3).

FIG. 22. Mean plasma concentration-time profile of ivacaftor following the sub-cutaneous administration of ivacaftor-loaded PLGA microparticles (formulation 1) to mice (n=30) at a dose of 50 mg/kg vs intra-venous administration of soluble ivacaftor (in a 10% Tween 80 in PBS, pH=7.4 vehicle) to mice (n=21) at a dose of 5 mg/kg. Error bars represent the standard deviation (±SD, n=3). PKsolver (an excel ad-in) was used to run non-compartmental PK analysis to estimate the AUC₀₋ₜ values of both groups.

FIG. 23. Images of the PLGA 75:25 Pellets (F7) in Example 8.

FIG. 24. In vitro release kinetics profile up to 14 days in two physiologically compatible mediums.

### DETAILED DESCRIPTION

Until recently, CF treatment has focused on the treatment of the complications of the disease. For example, antibiotics were used for lung infections, osmotic agents were used to decrease airway dehydration, and mucolytics, and bronchodilators like β₂ receptor agonists were used to clear airways (Deeks, 2016). Recently, a breakthrough in the field of CF treatment emerged after a new class of therapies known as CFTR modulators that target the underlying cause of the disease were approved in the last decade (Hoy, 2019). These pharmacological agents mainly target the two most common CFTR mutations, class II mutations (e.g. F508del, which involves defective trafficking, and occurs in 70% of CF patients), and class III mutations (e.g. G551D, which involves defective gating, and occurs in 5% of CF patients). The first of these agents to be approved was ivacaftor (VX-770, Kalydeco®, January 2012), which targets class III mutations by increasing the opening probability of CFTR channels at the cell surface (CFTR gate “potentiator). To further enhance CFTR protein activity, potentiators and correctors (those agents that increase CFTR protein trafficking to the cell surface, e.g. lumacaftor) may be used in combination (Hoy, 2019). In 2015, Orkambi®, the first fixed dose tablet combining a potentiator (ivacaftor) and a corrector (lumacaftor), has been approved for medical use in patients who are homozygous or heterozygous for class II CFTR mutation (F508del). In October 2019, the first approval was received for a triple combination therapy of the CFTR corrector tezacaftor, the next-generation CFTR corrector elexacaftor and the CFTR potentiator ivacaftor, under the brand name Trikafta®, for the treatment of CF patients who have one or more F508del mutation in the CFTR gene.

### I. Exemplary Composition Having a Carrier and Methods of Making and Using the Compositions

In one embodiment, the disclosure provides for a composition comprising one or more of lipophilic drugs, e.g., one or more CFTR modulators, and a carrier, which is useful, in one embodiment, for the treatment of cystic fibrosis. In one embodiment, the composition comprises therapeutic doses of the CFTR modulator(s). In one embodiment, the CFTR modulators include one of ivacaftor, lumacaftor, tezacaftor, or elexacaftor. In one embodiment, the carrier comprises cyclodextrin (CD), e.g., one or a binary or a ternary mixture of α-, β, γ, HP-β-CD, M-β-CD, or HP-γ-CD.

Cyclodextrins (CD's) are cyclic oligosaccharides formed of 6, 7, or 8 α-D-glycopyranose (i.e. dextrose) units, which are known as α, β, or γCD's, respectively. Due to their unique cyclic structures characterized by hydrophilic surfaces and hydrophobic cavities, they are able to host lipophilic molecules inside their cavities, forming supramolecular inclusion complexes. These complexes have been used to improve aqueous dissolution rate and oral bioavailablity of poorly water-soluble drugs, especially those that belong to BCS class II or IV drugs. Inclusion complexes involving cyclodextrins have also been employed to mask undesirable taste, improve drug stability, and reduce toxicity. While α, β, and γCD's are produced naturally by the enzymatic degradation of starch in bacteria, some other members are synthetic, including hydroxypropyl βCD (HPβCD), hydroxypropyl γCD (HPγCD), and methyl βCD (MβCD) (Hedges, 2009).

In one embodiment, the composition is formulated for oral administration. In one embodiment, oral administration is achieved after compression into tablets or suspension of the lyophilized powder composition into a suitable liquid oral vehicle. In one embodiment, the composition is formulated for subcutaneous, intravenous or intramuscular injection. In one embodiment, the use of the composition, e.g., therapeutic doses of the CFTR modulator, provides for therapeutic benefits in CF patients and/or any of its associated health manifestations, including respiratory distress, in utero CF-related abnormalities, and other diseases related or unrelated to CF that are known to be treated by the CFTR modulator(s) in human or animals. In one embodiment, the molar ratio between the CFTR modulator and the carrier (e.g., cyclodextrin) is 3:1 up to 1:10. In one embodiment, the composition includes one CFTR modulator in a carrier or a mixture of CFTR modulators in a mixture of carriers. In one embodiment, the composition comprises ivacaftor or any other CFTR modulator and a carrier that is a solubilizer to form a clear aqueous-based solution. In one embodiment, the solubilizer comprises polysorbate 80 (Tween 80). In one embodiment, the concentration of solubilizer, e.g., polysorbate 80, is 1-25% v/v. In one embodiment, the solubilizer comprises polysorbate 20, polysorbate 80, ethanol, polyethylene glycol, propylene glycol, or a mixture of these compounds.

In one embodiment, a method to prepare the composition is provided which comprises kneading of the CFTR modulator and the carrier by means of a mortar and a pestle in a solution comprising water and alcohol at ratios of 2:1 up to 1:2 followed by drying, e.g., the drying is achieved by vacuum, using any of the means known in the field, including lyophilization and others.

In one embodiment, a method to prepare the composition is provided by dissolving the CFTR modulator in a small amount of a compatible solvent (e.g., ethanol) and mixing it with an aqueous solution of the carrier under stirring. A powdered composition may be achieved by drying.

### A. OTHER FORMULATIONS

The disclosed compositions, e.g., having one or more CFTR modulators, may include or may be formed from biodegradable polymeric molecules which may include, but are not limited to polylactic acid (PLA), polyglycolic acid (PGA), co-polymers of PLA and PGA (e.g., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

Nanoparticles may be prepared by methods known in the art. (See, e.g., Nagavarma et al., Asian J. of Pharma. And Clin. Res., Vol 5, Suppl 3, 2012, pages 16-23; Cismaru et al., Rev. Roum. Chim., 2010, 55(8). 433-442; and International Application Publication Nos. WO 2012/115806; and WO 2012/054425; the contents of which are incorporated herein by reference in their entireties). Suitable methods for preparing the nanoparticles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the nanoparticles may be prepared by forming a double emulsion (e.g., water-in-oil-in-water) and subsequently performing solvent-evaporation. The nanoparticles obtained by the disclosed methods may be subjected to further processing steps such as washing and lyophilization, as desired. Optionally, the nanoparticles may be combined with a preservative (e.g., trehalose).

Typically, the nanoparticles have a mean effective diameter of less than 1 micron, e.g., the nanoparticles have a mean effective diameter of between about 25 nm and about 500 nm, e.g., between about 50 nm and about 250 nm, about 100 nm to about 150 nm, about 150 nm to about 175 nm, about 150 nm to about 200 nm, about 400 nm to about 450 nm, or about 450 nm to 650 nm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

Nanoparticles may have a zeta-potential that facilitates uptake by a target cell. Typically, the nanoparticles have a zeta-potential greater than 0. In some embodiments, the nanoparticles have a zeta-potential between about 5 mV to about 45 mV, between about 15 mV to about 35 mV, or between about 20 mV and about 40 mV. Zeta-potential may be determined via characteristics that include electrophoretic mobility or dynamic electrophoretic mobility. Electrokinetic phenomena and electroacoustic phenomena may be utilized to calculate zeta-potential.

In one embodiment, a composition comprising one or more CFTR modulators comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(p-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The disclosed polymer may include, but are not limited to, polyamidoamine (PAMAM) dendrimers. Polyamidoamine dendrimers suitable for preparing the presently disclosed nanoparticles may include 3rd-, 4th-, 5th-, or at least 6th-generation dendrimers.

In one embodiment, the delivery vehicle comprises a cationic lipid, e.g., N-[1-(2,3-dioleoyloxy)propel]-N,N,N-trimethylammonium (DOTMA), 2,3-dioleyloxy-N-[2-spermine carboxamide]ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N-dimethylaminoethane) carbamoyl] cholesterol (DC-Chol); dioctadecyl amidoglyceryl spermine (DOGS, Transfectam); or imethykdioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008: van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene. N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondroitin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide). N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Exemplary Carriers

In one embodiment, the carrier comprises cyclodextrin. In one embodiment the cyclodextrin is a naturally occurring cyclodextrin, e.g., one that improves solubility of a therapeutically active agent, such as alpha-cyclodextrin, beta-cyclodextrin or gamma-cyclodextrin, which when combined with the agent forms inclusion complexes. In one embodiment the cyclodextrin is a modified cyclodextrin, e.g., one that improves solubility of a therapeutically active agent, such as hydroxy-propyl beta cyclodextrin, hydroxy-propyl gamma cyclodextrin, methylated-beta cyclodextrin, sulfobutyl-ether-beta-cyclodextrin or O-carboxymethyl-O-ethyl-beta-cyclodextrin.

In one embodiment, the carrier comprises a surfactant. In one embodiment, the surfactant comprises an anionic surfactant, e.g., a surfactant comprising hydrophilic groups including but not limited to carboxylates, e.g., alkyl carboxylates- and their fatty acid salts or carboxylate-fluoro_surfactants; sulfates, e.g., alkyl sulfates (e.g., sodium lauryl sulfate); alkyl ether sulfates (e.g., sodium laureth sulfate); sulfonates, e.g., docusates (e.g., dioctylsodium sulfosuccinate); alkyl benzene sulfonates; or phosphate esters, e.g., alkylaryl ether phosphates, alkyl ether phosphates or sodium lauryl sulphate BP. In one embodiment, the surfactant comprises an cationic surfactant, e.g., a surfactant comprising hydrophilic groups including but not limited to RN+H₃Cl·(salt of a long-chain amine) or RN+ (CH₃)₃Cl·(quaternary ammonium chloride). In one embodiment, the surfactant comprises an zwitterionic surfactant, e.g., RN⁺H₂CH₂COO⁻, RN+(CH₃)₂CH₂CH₂ SO₃⁻, phospholipids or phosphatidylcholine (Lecithin). In one embodiment, the surfactant comprises a non-ionic surfactant, e.g., polyol esters, e.g., glycol, glycerol esters and sorbitan derivatives such as fatty acid esters of sorbitan and their ethoxylated derivatives including but not limited to sorbitan monolaurate, sorbitan monopalmitate sorbitan monostearate, sorbitan mono-oleate, sorbitan tristearate or sorbitan trioleate, or polyoxyethylene (20) sorbitan monolaurate, polyoxyethylene (20) sorbitan monopalmitate, polyoxyethylene (20) sorbitan monostearate, polyoxyethylene (20) sorbitan mono-oleate, polyoxyethylene (20) sorbitan tristearate, or polyoxyethylene (20) sorbitan tri-oleate.

### B. ROUTES OF ADMINISTRATION, DOSAGES AND DOSAGE FORMS

Administration of the compositions may be continuous or intermittent, depending, for example, upon the recipient's physiological condition, and other factors known to skilled practitioners. The administration of the composition(s) may be essentially continuous over a preselected period of time or may be in a series of spaced doses. Both local administration, e.g., intranasal or intrathecal, and systemic administration are contemplated. Any route of administration may be employed, e.g., intravenous, intranasal or intrabronchial, or local administration.

One or more suitable unit dosage forms comprising the composition(s), which may optionally be formulated for sustained release, can be administered by a variety of routes including parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the active agent with carriers, solid matrices, semi-solid carriers, finely divided solid carriers or combinations thereof, and then, if necessary, introducing or shaping the product into the desired delivery system.

The amount of composition(s) administered to achieve a particular outcome will vary depending on various factors including, but not limited to, the formulation, the condition, patient specific parameters, e.g., height, weight and age, and whether prevention or treatment, is to be achieved.

Compositions of the invention may conveniently be provided in the form of formulations suitable for administration. A suitable administration format may best be determined by a medical practitioner for each patient individually, according to standard procedures. Suitable pharmaceutically acceptable carriers (excipients) and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Compositions of the present invention may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The compositions can be provided in a dosage form containing an amount effective in one or multiple doses. For example, the active agent may be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results. The amount administered will vary depending on various factors including, but not limited to, the agent and/or carrier chosen for administration, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline.

For example, the composition/active agent(s) can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

Pharmaceutical formulations can be prepared by procedures known in the art using well known and readily available ingredients. For example, the therapeutic agent can be formulated with one or more common excipients, diluents, or carriers, and formed into tablets, capsules, suspensions, powders, and the like. The compositions of the invention can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the compositions may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the composition is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the composition may be administered via nose drops, a liquid spray, such as via a plastic bottle atomizer or metered-dose inhaler. Typical of atomizers are the Mistometer (Wintrop) and the Medihaler (Riker).

The local or systemic delivery can be by a variety of techniques, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### C. EXEMPLARY EMBODIMENTS

In one embodiment, a composition comprising one or more lipophilic CFTR modulators and a carrier is provided. In one embodiment, the carrier comprises a cyclodextrin. In one embodiment, the carrier comprises a non-ionic surfactant. In one embodiment, the mount of the modulator is therapeutically effective, e.g., sufficient to achieve therapeutic benefits in CF and/or any of its associated health manifestations, including respiratory distress, in utero CF-related abnormalities, and other diseases related or unrelated to CF that are known to be treated by the said pharmacological agents in human or animals. In one embodiment, the CFTR modulator may be ivacaftor, lumacaftor, tezacaftor, or elexacaftor. In one embodiment, the carrier comprises one or a binary or a ternary mixture of α-, β, γ, HP-β-CD, M-β-CD, or HP-γ-CD. In one embodiment, the carrier comprises a polysorbate. In one embodiment, the composition is formulated for oral administration, e.g., the composition is a tablet or a suspension of lyophilized powder CFTR modulator(s) in a suitable liquid oral vehicle. In one embodiment, the composition is formulated for subcutaneous or intramuscular injection. In one embodiment, the molar ratio between the CFTR modulator and the cyclodextrin is 3:1 to 1:10.

In one embodiment, a method to prepare the compositions comprises kneading of the CFTR modulator and the carrier by means of a mortar and a pestle by the help of a solution comprising water and alcohol at ratios of 2:1 to 1:2 followed by drying, e.g., the drying is achieved by vacuum, using any of the means known in the field, including lyophilization and others.

In one embodiment, a method to prepare the compositions comprises dissolving the CFTR modulator in a small amount of a compatible solvent (e.g. ethanol) and mixing it with an aqueous solution of the carrier under stirring. A powdered composition may be obtained by drying.

In one embodiment, the composition comprises a CFTR modulator and a carrier. In one embodiment, the composition comprises a mixture of CFTR modulators in a mixture of carriers. In one embodiment, the composition comprises ivacaftor or any other CFTR modulator and a carrier that is a solubilizer to form a clear aqueous-based solution. In one embodiment, the solubilizer is polysorbate 80. In one embodiment, the concentration of polysorbate 80 in the composition is 1-25% v/v. In one embodiment, the carrier that is a solubilizer may be, for example, polysorbate 20, polysorbate 80, ethanol, polyethylene glycol, propylene glycol, or a mixture of these compounds. In one embodiment, the composition is delivered by the oral route, intramuscular (IM) route, intravenous (IV) route, or subcutaneous (SC) mute. In one embodiment, the composition is administered in an amount that inhibits or treats one or more symptoms or abnormalities associated with of CF, including in a fetus.

As disclosed herein, compositions were made comprising ivacaftor or other CFTR modulators and one of the cyclodextrins, where the ivacaftor:CD molar ratio range was from 2:1 to 1:5. These compositions were made by co-grinding of constituents in the presence of a mixture of water and alcohol at a 1:1 v/v ratio at a sufficient amount (kneading method). Co-grinding continued for up to one hour, and the formed mixture was dried under vacuum. Another method enclosed involves dissolving the cyclodextrin in water and dissolving ivacaftor in the minimum amount of alcohol, followed by adding the alcoholic solution to the aqueous solution during stirring (solution method). The composition can be obtained following evaporation of the solvent using an oven, vacuum, or any other mean known to perform evaporation. In one embodiment, the type of cylcodextrin used was HPβCD and the drug:HPβCD molar ratio was 1:2 and the drying technique was lyophilization overnight. In one embodiment, the type of cyclodextrin used was γCD and the drug:γCD molar ratio was 1:2 and the drying technique was lyophilization overnight. In one embodiment, the final form of the formed composition is a powder. In one embodiment, the entrapped molecule is lumacaftor. In one embodiment, the entrapped molecule is tezacaftor. In one embodiment, the entrapped molecule is elexacaftor.

Also provided is a method of administering such compositions. A human or an animal may be administered these compositions by, for example, the oral route, either after suspending the powder in a sufficient amount of water or another palatable liquid, or after compressing these powders into tablets using common tableting excipients. In one embodiment, these compositions are to be given orally to animals (for example mice, rats, ferrets, sheep, cows, etc.) or humans that carry mutations responsive to CFTR modulators. In one embodiment, these compositions can be given to pregnant animals carrying fetuses that have a mutation that responds to CFTR modulators. In one embodiment, these compositions are given orally to mice. In one embodiment, these compositions are given to pigs. In one embodiment, these compositions are injected subcutaneously.

These compositions also unexpectedly have higher oral bioavailability compared to a commercial product with the same active ingredient, prolonged plasma levels when given orally, higher liver levels in the fetuses of pregnant experimental animals with CFTR-G551D mutation, and resulted in the birth of piglets with relatively normal pancreas, intestine, and gall bladder, from pregnant animals with the mutation.

Prior to the preparation of such compositions, phase solubility studies of ivacaftor in different molar concentrations of cyclodextrins was carried out. It was unexpectedly found that the drug solubility in different molar ratios of βCD was about 5-6 times higher than its counterparts in different molar concentrations of αCD and γCD. Furthermore, it was unexpectedly found that the drug solubility in HPβCD was about 100-150 times higher than that in similar concentrations of αCD and γCD (50 μM of each CD), and about 40 times higher than that in the maximum concentration tested for βCD (10 μM), considering the limited aqueous solubility of βCD. Ivacaftor dissolved in a 50 μM solution of HPβCD at a concentration of 50 μg/ml, which is about 1000 times higher than its aqueous solubility (about 0.05 μg/ml) at room temperature.

### II. Exemplary Microparticle Compositions and Methods of Use

An existing formulation of ivacaftor that is currently available in the market is in the form of oral tablets (Kalydeco®). This is an amorphous solid dispersion formulation intended to improve the solubility and bioavailability of ivacaftor following oral administration. The disadvantage of this formulation is that the patient is required to administer 2 tablets/day (150 mg each) for ivacaftor to reach enough therapeutic levels in the blood. Since cystic fibrosis (CF) is a long-term disease, this could be inconvenient for patients specially that CF patients usually administer multiple medications to manage the disease's symptoms. This underlines the need to develop an injectable drug delivery system capable of improving IVA's bioavailability while allowing its sustained release to eliminate the need of its frequent twice daily oral administration to ultimately improve patient compliance and convenience and CF treatment outcome.

The present disclosure provides for a long-acting particle formulation that is intended to provide steady plasma levels of a lipophilic drug such as ivacaftor (IVA) over a period of one month following a single sub-cutaneous injection and thus avoid the need for frequent administration of the drug. This can improve the patient's quality of life and avoid the unwanted toxic or sub-therapeutic effects that could result from multiple administrations of a drug. In one embodiment, subcutaneous administration of the formulation (1 mg of ivacaftor) resulted in mean plasma levels of ivacaftor ranging from 0.05 to 1 μg/mL which is in contrast to oral administration of 150 mg ivacaftor which resulted in mean plasma levels ranging from 0.5 to 1 μg/mL In one embodiment, IVA was encapsulated into an injectable, biodegradable, long-acting poly(D,L-lactide-co-glycolide) (PLGA) particle formulation, e.g., to overcome the need for its frequent twice daily oral administration by providing a sustained release of the drug and hence improve patient convenience and compliance to treatment. A number of polymers, besides PLGA, have been reported for delivering drugs and cancer vaccines, such as: poly(diaminosulfide), polyanhydride and polycaprolactone (PCL) (Wafa et al., 2017; Wafa et al., 2019; Wafa et al., 2019b; Nair et al., 2007; Ebeid et al., 2018; Ahmed et al., 2020). PLGA (FIG. 17) is a synthetic, polyester-based, bulk-eroding co-polymer that is synthesized by the copolymerization of lactic and glycolic acid monomers. It is a biodegradable (hydrolytically and enzymatically degradable) and biocompatible copolymer that is accepted for human use by the US Food and Drug administration (FDA) and the European Medicines Agency (Nair et al., 2007; Quartman et al., 20201 Han et al. 2016, Bahareh et al., 2014). It has been the most widely used synthetic copolymer for injectable long-acting formulations for more than 3 decades, whether in microparticles, in situ forming implants or solid implants. It is mainly used to encapsulate small molecules (hydrophilic and hydrophobic), proteins, peptides, and antigens (as adjuvants in cancer vaccines and immunotherapy) (Tushi et al., 2013; Naguib et al., 2020: Naguib et al., 2020b).

One of the advantages of using PLGA in developing long-acting drug delivery systems is the ability to tailor the release profile of the encapsulated drug by controlling the polymer chemistry. This includes the polymer's lactide:glycolide ratio (L:G ratio), molecular weight, and end-group functionalization (Lagreca et al., 2020) In addition, long-acting formulations avoid toxic or subtherapeutic effects that can result from repeated administration of drugs due to the sharp fluctuations in plasma drug concentrations, by providing steady plasma levels overtime (Naguib et al., 2020; Park et al., 2020; Park et al., 2019). Porsio et al. (2018, 2020) and Zhu et al. (2020) have recently encapsulated IVA into inhalable nano-into-micro dry powders, nanocomposite microparticles as well as mucus penetrating nanoparticles for the goal of enhancing its pulmonary delivery.

As described herein below, to develop a particle formulation with defined characteristics (e.g., particle size, drug loading, encapsulation efficiency and/or drug release kinetics), the effect of various formulation parameters on the produced particles' characteristics was assessed. One of the formulations was injected sub-cutaneously in mice to study its pharmacokinetics. In addition, IVA was dissolved in an FDA-approved vehicle (10% v/v polysorbate 80 in sterile phosphate buffered saline (PBS), pH 7.4) and injected it intra-venously in mice to compare its pharmacokinetics with that of the sub-cutaneously injected IVA particles. Thus, IVA was encapsulated in an injectable, long acting, PLGA-based microparticle formulation and its' in vitro release kinetics and in vivo pharmacokinetics was compared to that of soluble IVA in mice.

Six injectable long-acting ivacaftor-loaded microparticle formulations were developed using poly(D,L-lactide-co-glycolide) (PLGA) polymer. Sustained-release microparticle formulations carry many advantages including their ability to provide steady plasma levels of the encapsulated drug and avoid sharp fluctuations of the drug concentrations in the plasma that result from repeated administration of immediate release formulations. These sharp fluctuations can result in either toxic or sub-therapeutic concentrations of the drug (Ikpa et al., 2014). Using long-acting microparticle formulations, the drug levels in the plasma are maintained within the therapeutic window of the drug over a long period of time following a single sub-cutaneous or intra-muscular injection (Condren et al., 2013). This not only avoids adverse effects or unsuccessful treatment outcomes that may result from frequent administration of the drug, but also significantly improves patient's convenience and willingness to adhere to the treatment due to administering the microparticle formulation at a much lower frequency over the course of treatment and result in treatment success. To develop the ivacaftor-loaded microparticle formulation we used poly(D,L-lactide-co-glycolide) (PLGA) polymer, which is a biodegradable and biocompatible polymer that is generally recognized as safe (GRAS) by the United States Food and Drug Administration (FDA) (Fohner et al., 2017; Deek, 2016; Conway et al., 1996).

Besides having multiple applications in the market such as in surgical sutures, implants and prosthetic devices, PLGA can be used to tailor the release of the encapsulated drug in the microparticles by controlling the ratio of lactide to glycolide monomers (L:G ratio) in the polymer (Wafa et al., 2017; Wafa et al., 2019). This advantage makes PLGA an attractive polymer to use for encapsulating drugs in safe, injectable, long-acting microparticle formulations.

### A. EXEMPLARY FORMULATIONS

In one embodiment, the formulation comprises particles comprising one or more CFTR modulators. The disclosed particles, e.g., biodegradable microparticles, may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The disclosed particles may be prepared by methods known in the art. (See, e.g., Nagavarma et al., Asian J. of Pharma. And Clin. Res., Vol 5, Suppl 3, 2012, pages 16-23; Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing particles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the particles may be prepared by forming a double emulsion (e.g., water-in-oil-in-water) and subsequently performing solvent-evaporation. The particles may be subjected to further processing steps such as washing and lyophilization, as desired. Optionally, the particles may be combined with a preservative (e.g., trehalose).

In one embodiment, the particles have a mean effective diameter of less than 500 microns, e.g., the particles have a mean effective diameter of between about 1 μm and about 500 μm, e.g., between about 5 μm and about 25 μm, about 10 μm and about 20 μm, about 15 μm and about 25 μm, about 100 μm to about 150 μm, or about 45 μm to 650 μm. In one embodiment, the particles have a mean effective diameter of less than 50 microns, e.g., the particles have a mean effective diameter of between about 0.01 μm and about 50 μm, e.g., between about 0.5 μm and about 5 μm, about 1 μm and about 10 μm, about 1 μm and about 7.5 μm, about 5 μm to about 10 μm, about 4 μm to about 7 μm or about 2 μm to about 5 μm.

In one embodiment, the particles have a mean effective diameter of less than 1.5 mm, e.g., the particles have a mean effective diameter of between about 1 mm and 1.4 mm, about 0.5 mm to about 1 mm, about 0.6 mm to about 0.8 mm, e.g., 0.7 mm, about 0.1 to about 0.5 mm.

The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

In one embodiment, the particles comprise polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to liposomes, emulsions, DOTAP, DOTMA, DMRIE, DOSPA, distearoylphosphatidylcholine (DSPC), DOPE, or DC-cholesterol; peptide based vectors including but not limited to poly-L-lysine or protamine; or poly(O-amino ester), chitosan, PEI-polyethylene glycol, PEI-mannose-dextrose, DOTAP-cholesterol or RNAiMAX.

In one embodiment, the particle is a glycopolymer-based particle, poly(glycoamidoamine)s (PGAAs). These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units may yield exceptional delivery efficiency.

In one embodiment, the particles comprise polyethyleneimine (PEI), polyamidoamine (PAMAM), PEI-PEG, PEI-PEG-mannose, dextran-PEI. OVA conjugate, PLGA microparticles, or PLGA microparticles coated with PAMAM, or any combination thereof. The polymer may include, but is not limited to, polyamidoamine (PAMAM) dendrimers. Polyamidoamine dendrimers suitable for preparing the particles may include 3rd-, 4th-, 5th-, or at least 6th-generation dendrimers.

In one embodiment, the delivery vehicle may be particles or liposomes comprising a cationic lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-p-[N—(N,N-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged molecules by means of electrostatic interaction to slightly positively charged particles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of polymers include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although other materials, for example, PEI, DOTMA, DC-Chol, or CTAB, may be used.

In one embodiment, the particles comprise hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene. N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondroitin sulfate, and microbial polyesters, e.g., hydroxyalkanoates such as hydroxyvalerate and hydroxybutyrate copolymers, and synthetic polymers. e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM may be from a combination of sources. Isolated ECM may be prepared as a sheet, in particulate form, gel form and the like.

The biocompatible polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### B. PHARMACEUTICAL COMPOSITIONS

The disclosure provides a composition comprising, consisting essentially of, or consisting of microparticles, particles or liposomes comprising one or more CFTR modulators and optionally a pharmaceutically acceptable (e.g., physiologically acceptable) carrier. In one embodiment, additional components can be included that do not materially affect the composition (e.g., adjuvants, buffers, stabilizers, anti-inflammatory agents, solubilizers, preservatives, etc.). In one embodiment, when the composition consists of the polymer or particles formed therefrom, the modulator and optionally the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the CFTR modulator is administered in a composition formulated to protect the CFTR modulator from damage prior to administration. In addition, one of ordinary skill in the art will appreciate that the CFTR modulator can be present in a composition with other therapeutic or biologically-active agents.

Injectable depot forms are envisioned including those having biodegradable polymers such as polylactide-polyglycolide. Depending on the ratio of inhibitor to polymer, and the nature of the particular polymer employed, the rate of inhibitor release can be controlled. Examples of other biodegradable polymers include poly(orthoesters) and poly(anhydrides). Depot injectable formulations are also prepared by entrapping the inhibitor optionally in a complex with a polymer in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins. polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the CFTR modulator in the composition administered to the mammal will depend on a number of factors, including the size (mass) of the mammal, the extent of any side-effects, the particular route of administration, and the like. In one embodiment, the method comprises administering a “therapeutically effective amount” of the composition. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the MEK1/2 inhibitor to elicit a desired response in the individual. One of ordinary skill in the art can readily determine an appropriate CFTR modulator dose range to treat a patient having a particular disease or disorder, based on these and other factors that are well known in the art.

In one embodiment, the composition is administered once to the mammal. It is believed that a single administration of the composition may result in persistent expression in the mammal, optionally with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of the CFTR modulator as described above.

### C. ROUTES OF ADMINISTRATION, DOSAGES AND DOSAGE FORMS

Administration of the CFTR modulator may be continuous or intermittent, depending, for example, upon the recipient's physiological condition, and other factors known to skilled practitioners. The administration of the MEK1/2 inhibitor may be essentially continuous over a preselected period of time or may be in a series of spaced doses. Both local administration, e.g., intranasal or intrathecal, and systemic administration are contemplated. Any route of administration may be employed, e.g., intravenous, intranasal or intrabronchial, or local administration. In one embodiment, compositions may be subcutaneously, orally or intravascularly delivered.

One or more suitable unit dosage forms comprising the CFTR modulator, which may optionally be formulated for sustained release, can be administered by a variety of routes including local, e.g., intrathecal, oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the CFTR modulator with liquid carriers, solid matrices, semi-solid carriers, finely divided solid carriers or combinations thereof, and then, if necessary, introducing or shaping the product into the desired delivery system.

The amount of the CFTR modulator administered to achieve a particular outcome will vary depending on various factors including, but not limited to the condition, patient specific parameters, e.g., height, weight and age, and whether prevention or treatment, is to be achieved.

The CFTR modulator may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The microparticles containing one or more CFTR modulators may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The CFTR modulator can be provided in a dosage form containing an amount effective in one or multiple doses. The CFTR modulator may be administered in dosages of at least about 0.0001 mg/kg to about 20 mg/kg, of at least about 0.001 mg/kg to about 0.5 mg/kg, at least about 0.01 mg/kg to about 0.25 mg/kg, at least about 0.1 mg/kg to about 0.25 mg/kg of body weight, about 0.1 mg/kg to about 0.5 mg/kg, about 0.5 mg/kg to about 2 mg/kg, about 1 mg/kg to about 5 mg/kg, about 5 mg/kg to about 10 mg/kg, or about 10 mg/kg to about 20 mg/kg although other dosages may provide beneficial results. For example, a daily dose of ivacaftor for a human may range from 1 mg/kg to 10 mg/kg, e.g., 3 mg/kg to 5 mg/kg including 4.2 mg/kg (for a 70 kg human), of Orkambi (Lumacaftor/ivacaftor) may be from 2 mg/kg to 7 mg/kg, including 4 mg/kg to 6 mg/kg or 5.2 mg/kg (for a 70 kg human) lumacaftor and 0.5 mg/kg to 3 mg/kg, e.g., 1 mg/kg to 20 mg/kg or 1.7 mg/kg (for a 70 kg human) ivacaftor, of Symedko (Tezacaftor/ivacaftor) is 0.5 mg/kg to 4 mg/kg, e.g., 1 mg/kg to 3 mg/kg or 2.5 mg/kg (for a 70 kg human) tezacaftor and 1 mg/kg to 6 mg/kg, e.g., 2.5 mg/kg to 5 mg/kg or 4.2 mg/k for a 70 kg human) of ivacaftor, or of elexacaftor/tezacaftor/ivacaftor may be from 0.5 mg/kg to 4 mg/kg, e.g., 1 mg/kg to 3 mg/kg or 2.8 mg/kg (for a 70 kg human) of elexacaftor, 0.3 mg/kg to 3 mg/kg, e.g., 1 mg/kg to 2 mg/kg or 1.4 mg/kg (for a 70 kg human) of tezacaftor and 1.0 mg/kg to 5 mg/kg. e.g., 2 mg/kg to 4 mg/kg or 3.2 mg/kg (for a 70 kg human) ivacaftor

The amount administered will vary depending on various factors including, but not limited to, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. In one embodiment, from 0.0001 to 1 mg or more, e.g., up to 1 g, in individual or divided doses, e.g., from 0.001 to 0.5 mg, or 0.01 to 0.1 mg, of CFTR modulator can be administered.

Pharmaceutical formulations containing the CFTR modulator can be prepared by procedures known in the art using well known and readily available ingredients. For example, the agent can be formulated with common excipients, diluents, or carriers, and formed into tablets, capsules, suspensions, powders, and the like. The CFTR modulator containing particles can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the CFTR modulator containing particle may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the CFTR modulator composition is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the CFTR modulator composition may be administered via nose drops, a liquid spray, such as via a plastic bottle atomizer or metered-dose inhaler. Typical of atomizers are the Mistometer (Wintrop) and the Medihaler (Riker).

The local delivery of the CFTR modulator composition can also be by a variety of techniques which administer the CFTR modulator composition at or near the site of disease, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### C. EXEMPLARY EMBODIMENTS OF PARTICLES

To develop the CFTR modulator-loaded particle formulations, a simple oil-in-water (o/w) single emulsion solvent evaporation technique may be employed for modulators with the hydrophobic nature of ivacaftor (log P=3.13). Briefly, 50 mg of the PLGA polymer and 5 or 10 mg CFTR modulator were dissolved in 1.5 mL dichloromethane (DCM) (organic phase). Dichloromethane (DCM) was chosen as solvent due to its ability to solubilize all kinds of PLGA regardless of the lactide:glycolide monomer ratio, in addition to its low boiling point (39.8° C.) which allows fast evaporation (Condren et al., 2014; Wafa et al., 2019). The organic solution was then added to 30 mL (1 or 1.5% w/v) polyvinyl alcohol (PVA) solution and the mixture was immediately emulsified for 1 min at (6500 or 13500 rpm) at room temperature using an overhead homogenizer (Ultra-turrax T25 basic, Ika Works, Inc., Wilmington, NC). Polyvinyl alcohol was used as an emulsifier due to its superior chemical and physical properties such as being non-toxic, biodegradable, biocompatible and stable towards temperature variation (Nair et al., 2007). The emulsion was then stirred using a magnetic digital stirrer at 25° C. set at speed 300 rpm for 2 h to evaporate DCM. To collect the particles, the suspension was centrifuged at 1000×g for 5 min (Eppendorf centrifuge 5864 R, Eppendorf North America, Hauppauge, NY). The collected particles were resuspended in 30 mL nanopure water (Barnstead Thermolyne Nanopure water purification system, Thermo Fisher, Waltham, MA), washed twice at the same centrifugation conditions to remove any remaining PVA or unencapsulated drug. Finally, the particles were resuspended in 1 mL nanopure water, frozen at −80° C. and lyophilized overnight at 0.045 mbar and a collector temperature of −105° C. (Labconco Free zone 4.5 L-105° C., Labconco, Kansas City, MO). After 24 h of lyophilization, particles were collected and stored at −20° C. until use.

To obtain an CFTR modulator-loaded particle formulation with specific formulation characteristics (particle size, drug loading, encapsulation efficiency, drug release kinetics), the effect of varying four formulation parameters on the produced particle formulation characteristics was studied. The formulation parameters varied are presented in Table A. The effect of varying the formulation parameters on the CFTR modulator-loaded mparticles formulations characteristics are shown in Table B.

TABLE A
Formulation parameters of six CFTR modulator-
loaded PLGA particle formulations.

| | CFTR | | | | | | | |
|---|---|---|---|---|---|---|---|---|
| | modulator | Polymer (PLGA) | | Emulsifier | | Emulsification | | Varied |
| | Amount | | Amount | | Conc. | Speed | Time | formulation |
| F# | (mg) | Type | (mg) | Type | (%) | (rpm) | (min) | parameter |
| 1 | 5 | Resomer ® | 50 | PVA | 1 | 6500 | 1 | Drug:polymer |
| | | RG 503 H | | | | | | ratio |
| 2 | 10 | Resomer ® | 50 | PVA | 1 | 6500 | 1 | |
| | | RG 503 H | | | | | | |
| 3 | 5 | Resomer ® | 50 | PVA | 1.5 | 6500 | 1 | Emulsifier |
| | | RG 503 H | | | | | | concentration |
| 4 | 5 | Resomer ® | 50 | PVA | 1 | 9500 | 1 | Emulsification |
| | | RG 503 H | | | | | | speed |
| 5 | 5 | Resomer ® | 50 | PVA | 1 | 13500 | 1 | |
| | | RG 503 H | | | | | | |
| 6 | 5 | Resomer ® | 50 | PVA | 1 | 6500 | 1 | Polymer |
| | | RG 502 H | | | | | | molecular |
| | | | | | | | | weight |

Resomer RG 503 H: PLGA (50:50) Mw (24,000-38,000) acid terminated
Resomer RG 502 H: PLGA (50:50) Mw (7,000-17,000) acid terminated
PVA: Polyvinyl alcohol

TABLE B
Characteristics of six CFTR modulator-loaded PLGA particle formulations

| | Drug | Encapsulation | | | | | Average | |
|---|---|---|---|---|---|---|---|---|
| | loading | efficiency (EE) | Yield | D₁₀ | D₅₀ | D₉₀ | diameter | Span |
| F# | % (w/w ± SD)¹ | % (w/w ± SD)¹ | % (w/w ± SD)¹ | (μm) | (μm) | (μm) | (μm ± SD)¹ | value |
| 1 | 8.25 ± | 90.7 ± | 48.5 ± | 5.26 | 6.83 | 8.3 | 6.83 ± | 0.28 |
| | 3.73 | 41.1 | 8.96 | | | | 1.18 | |
| 2 | 10.3 ± | 56.7 ± | 58.3 ± | 5.45 | 6.71 | 8.61 | 6.93 ± | 0.35 |
| | 6.6 | 36.3 | 2.21 | | | | 1.31 | |
| 3 | 4.48 ± | 49.3 ± | 61.4 ± | 5.38 | 6.36 | 7.38 | 6.38 ± | 0.19 |
| | 0.7 | 7.71 | 10.5 | | | | 0.77 | |
| 4 | 3.91 ± | 43 ± | 70.3 ± | 2.6 | 3.28 | 3.99 | 3.32 ± | 0.26 |
| | 0.37 | 4.09 | 9.15 | | | | 0.58 | |
| 5 | 2.59 ± | 26.6 ± | 63.6 ± | 1.47 | 1.9 | 2.43 | 1.91 ± | 0.36 |
| | 0.88 | 9.71 | 15.3 | | | | 0.41 | |
| 6 | 5.84 ± | 100 ± | 64.1 ± | 4.26 | 5.51 | 7.19 | 5.64 ± | 0.39 |
| | 3.55 | 39.1 | 5.9 | | | | 1.12 | |

¹Mean ± standard deviation (SD, n = 3)

The produced CFTR modulator-loaded particles may have an average diameter that ranged from 1.91-6.93 Nm with narrow size distributions shown by span values ranging from 0.19-0.39. Generally, a homogenous distribution of the particle sizes is observed. The particles may be smooth, non-porous, and/or spherical shape with no unencapsulated drug crystals.

The effect of varying different formulation parameters (drug:polymer ratio, surfactant concentration, emulsification speed, PLGA molecular weight) on the drug loading, encapsulation efficiency and release of a CFTR modulator from the PLGA spheres is evaluated.

a—Effect of Drug: Polymer Ratio (D:P)

Formulation 2 (F2), when IVA is employed, with a higher initial added drug amount (20%) had a higher average drug loading (±SD) of 10.3% (±6.6) vs 8.25% (±3.73) for F1. The higher drug loading in F2 resulted in a weaker average initial burst release (±SD) of 14.14% (±8.77) vs 23.73% (±5.6) in F1 after 3 days, and an overall slower % cumulative drug release.

In contrast to drug loading, F2 had a lower average encapsulation efficiency (±SD) of 56.7% (±36.3) in comparison to F1 with 90.7% (±41.1).

b—Effect of Surfactant Concentration

In formulation 3 (F3), IVA is employed with a higher surfactant concentration (1.5% PVA) than that used in F1 (1%).

Increasing the surfactant concentration has led to a decrease in particle size.

Increasing surfactant concentration resulted in lowering both DL and EE. F3 had an average DL (±SD) and EE (±SD) of 4.48% (±0.7) and 49.3% (±7.71) vs 8.25% (±3.73) and 90.7% (±41.1) for formulation 1 (F1), respectively.

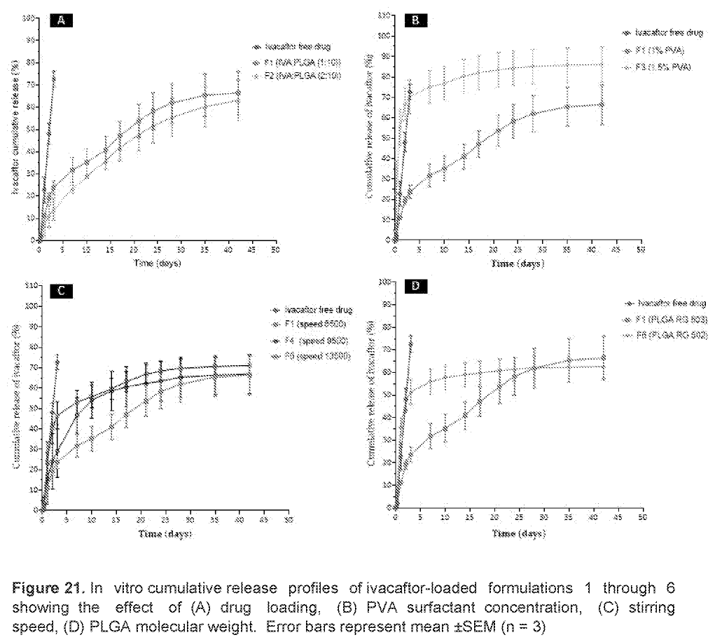

The release profiles of F1 and F3 with IVA are shown in FIG. 21. F3 (with smaller particle size) had a stronger initial burst release and an overall faster cumulative release rate than of F1. After 3 days, 70% (±14.3) of ivacaftor has been released from F3 vs only 23.7% (±5.6) from F1, and by 6 weeks, F1 has released 66.4% (±16.89) of the loaded ivacaftor vs 86.1% (±14.7) released from F3.

C— Effect of Stirring Speed:

The emulsification speed tested for IVA loaded particles was 6500 rpm (F1), 9500 (F4) and 13500 (F5).

F1 (with the lowest emulsification speed) had an average particle size diameter (±SD) of 6.83 μm (±1.18) compared to 3.32 μm (±0.58) and 1.91 μm (±0.41) for formulations 4 and 5, respectively. Higher stirring speed has resulted in smaller particle sizes, and as a result lower DL, EE and faster cumulative release profiles.

Formulation 1 had average DL (±SD) and EE (±SD) of 8.25% (±3.73) and 90.7% (±41.1) compared to 3.91% (±0.37) and 43% (±4.09) for formulation 4, 2.59% (±0.88) and 26.6% (±9.71) for formulation 5, respectively.

FIG. 21c shows the in vitro cumulative release profiles of formulations 1, 4, and 5 with IVA. After 3 days, formulation 1 have released (±SD) 23.7% (±0.44) vs 29.13% (±22.41) from formulation 4 and 46.63% (±11.53) from formulation 5. By 10 days, 35.24% (±10.4) of ivacaftor have been released from formulation 1 vs 54.1% (±15.3) and 55.8% (±9.4) released from formulations 4 and 5, respectively. And finally at the end of the release study (6 weeks), 66.46% (±16.8) of ivacaftor have been released from formulation 1 vs 66.9% (±16.5) and 71.1% (8.1) released from formulations 4 and 5, respectively.

d—Effect of Polymer Molecular Weight

In formulation 6 (F6), a polymer of lower molecular weight (mol.wt.) and inherent viscosity than that used in formulation 1 (F1) was tested with IVA. The polymers used in F1 and F6 were RG503H and RG502H with mol.wt. ranges from 24,000-38,000 and 7,000-17,000 and inherent viscosities of 0.32-0.44 and 0.16-0.24 dL/g in chloroform, respectively.

Decreasing the polymer molecular weight has resulted in microparticles with a smaller average diameter 5.64 um (±1.12) in F6 vs 6.83 um (±1.18) in F1, as described above. As a result, F6 had a lower average DL (±SD) of 5.84% (±3.55) vs 8.25% (±3.73) in F1.

Lowering the polymer molecular weight in F6 resulted in a stronger initial burst release and an overall faster release rate specially during the first 4 weeks. After 3 days, 23.7% (±5.6) and 51.4% (±9.46) of the encapsulated drug has been released from F1 and F6, respectively (FIG. 21D).

This trend was reversed after the 30-day mark, where F1 had a slightly faster release than F6, releasing 66.4% (±16.9) and 62.6% (±8.7) by day 42, respectively.

Based on the particles characterization data (particle size, drug loading, encapsulation efficiency) and in vitro release kinetics. Formulation 1 (F1) has good drug loading and encapsulation efficiency values (±SD) of 8.25 (±3.73) and 90.7 (±41.1). In addition, to an appropriate particle size (±SD) of 6.83 μm (t 1.18) making it injectable and the in vitro release profile was characterized by a small burst release and an overall slow release as shown in FIG. 21A.

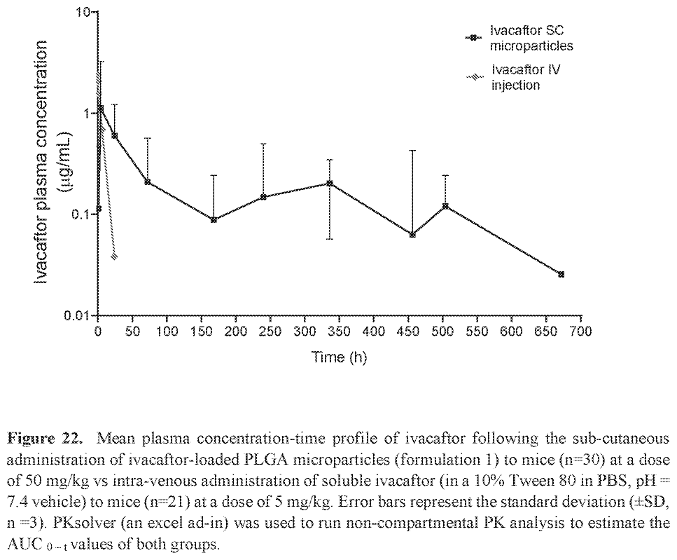

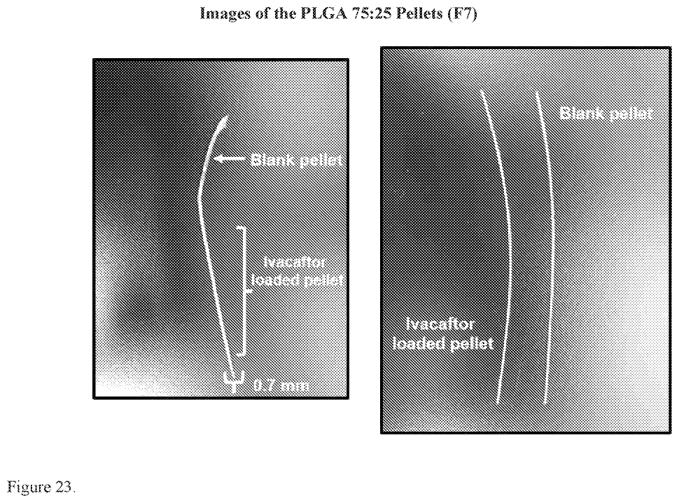

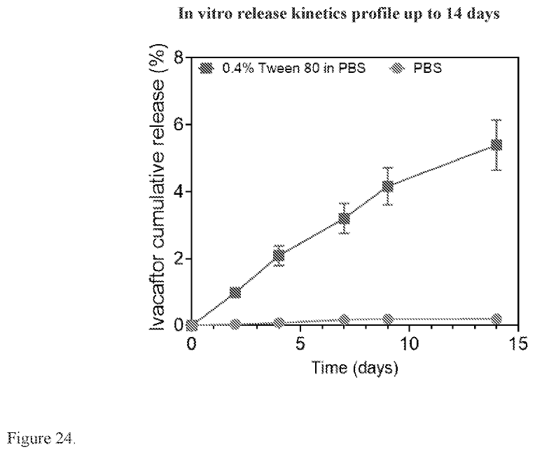

The in vivo plasma levels of ivacaftor following the intra-venous administration of 5 mg/kg solubilized ivacaftor and 50 mg/kg ivacaftor particles (formulation 1) to mice are shown in FIG. 22.

Sustained mice plasma levels of ivacaftor were observed up to 28 days following the SC administration of ivacaftor particle formulation 1 with concentrations ranging from 0.02-1.12 μg/mL. In contrast to the rapid elimination of ivacaftor following its intravenous administration to mice, where the last detectable plasma concentration was after only 1 day of administration. The area under the curve (AUC_{0-t) estimates resulting from non-compartmental pharmacokinetic analysis (NCA) of the ivacaftor mice plasma data following the administration of} 0.1 mg soluble ivacaflor and 1 mg ivacaftor microparticle formulation 1 has shown an approx. 6-fold increase in exposure reflected in AUC₀₋₄ values of 71.6 and 12.3 (μg/mL*h) for ivacaftor particles and soluble ivacaftor, respectively. These results indicate that the SC administration of ivacaftor particles can eliminate the need of frequent administration of ivacaftor to treat CF by providing sustained plasma levels over a long period of time (28 days).

### III. SUBJECTS

The subject may be any animal, including a human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, childrens, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially nor them European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

The invention will be described by the following non-limiting examples.

### Example 1

To test whether ivacaftor aqueous solubility is enhanced in presence of different molar concentrations of CD's the following experiments were conducted.

### Methods

Excess amounts of powdered ivacaftor were suspended in different concentrations of different types of CD's (up to 10 μM in the case of βCD, and up to 50 μM in the case of αCD, γCD, and HPβCD). The suspensions were shaken at 3000 rpm at room temperature for 3 days. Then, samples were collected and filtered, and the concentration of ivacaftor was determined using HPLC, as described below.

### HPLC

An Agilent HPLC workstation series 1100 (Agilent Technologies, Santa Clara, CA) was used for the analysis. The column used was Waters Symmetry C18 5 μm (4.6×150 mm), and the mobile phase consisted of 6:4 acetonitrile: water with 0.1% v/v trifluoroacetic acid (TFA) at a flow rate of 1 ml/min (at 25±2° C.). Detection was carried out using an Agilent Diode array detector (DAD) at 309 nm.

### Results

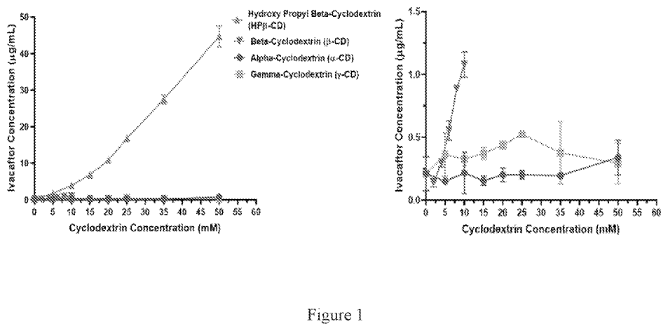

The solubility of ivacaftor in 10 μM of βCD was about 5-8 times higher than its counterparts in the same concentration of αCD and γCD solutions. Ivacaftor solubility in 50 μM HPβCD was about 100-150 times higher than those in similar concentrations of αCD and γCD, and about 40 times higher than that in the maximum concentration tested for βCD (10 μM), considering the limited aqueous solubility of βCD. Ivacaftor dissolved in a 50 μM solution of HPβCD at a concentration of 50 μg/ml (FIG. 1), which is about 1000 times higher than its aqueous solubility (about 0.05 μg/ml).

In addition, ivacaftor dissolved in a 25 μM solution of γCD at a concentration of 0.5 μg/ml, which is about 10 times its aqueous solubility. Also, the drug dissolved in a 10 μM solution of βCD at a concentration of 1.2 μg/ml, which is about 24 times its aqueous solubility (FIG. 1).

### Conclusion

Enhancement of ivacaftor solubility in CD's solutions may be attributed to non-covalent interaction (e.g. hydrogen bond formation), which makes ivacaftor a suitable candidate to form highly water soluble/dispersible inclusion complexes with CDs.

### Example 2

To prepare and characterize inclusion complexes comprising ivacaftor and HPβCD, the following methods were employed.

### Methods

The compositions were prepared using the kneading method mentioned earlier. The final lyophilized powdered formulation was characterized using differential scanning calorimetry (DSC), Fourier transform infrared spectroscopy (FT-IR, Perkin Elmer, Waltham MA)), powder x-ray diffraction (PXRD) and scanning electron microscopy (SEM). Preliminary dissolution profile was examined and compared to that of ivacaftor powder and a commercial ivacaftor product (Kalydeco®). For the DSC profiles evaluation, weighed amounts of ivacaftor. HPβCD, physical mixture of the two, and ivacaftor/HPβCD complex (molar ratio 1:2) were transferred into aluminum pans, and the pans were crimp-sealed. The DSC thermograms were obtained using a DSC instrument (TA Instruments model Q20, New Castle, DE) using a temperature ramp rate of 10° C./min, within a range of 25 to 350° C.

The X-ray powder diffraction patterns of each sample were obtained using a Siemens D5000 diffractometer. A Cu Kα X-rays with λ=1.51418 Å was used as X-ray source, Diffractograms were recorded in the range 5° to 50° at 2Θ values using a step size of 0.02° and a dwell time of 0.5 s. The diffractograms of pure ivacaftor and HP-β-CD, were compared with the physical mixture and inclusion complex to evaluate the interaction between ivacaftor and HP-β-CD.

The surface morphology of each sample was examined using a Hitachi S-4800 scanning electron microscope (SEM) (Hitachi High Technologies, Ontario, Canada). Samples were spread onto a carbon double-adhesive tape that is mounted on an aluminum stub, and the surface was coated with gold and palladium (to make the samples electrically conductive) using an argon beam K550 sputter coater (Emitech Ltd., Kent, U.K.). SEM photomicrographs were captured at 1.5 kV accelerating voltages

The FT-IR spectra of ivacaftor, hyroxypropyl beta-cyclodextrin (HPβCD), and ivacaftor/HPβCD complex (molar ratio 1:2) were recorded using a Perkin Elmer FTIR spectrometer (Waltham MA) using the KBr disc method. Spectra were recorded between 4000 and 500 cm⁻¹ at a resolution 0.5 cm⁻¹. In order to check the interaction between ivacaftor and HPβCD, the spectra of pure ivacaftor and pure HPβCD were compared with that of the ivacaftor/HPβCD complex.

Dissolution study was performed in 500 mL deionized water (containing 0.002% Polysorbate 80) at 37° C. using a U.S. Pharmacopeia (USP) Apparatus 2 with paddle rotating at 50 rpm. The dissolution behavior of pure ivacaftor powder, commercial ivacaftor product (Kalydeco®) and ivacaftor/HPβCD complex (molar ratio of 1:2) were compared by adding amounts equivalent to 2 mg ivacaftor from each sample into the dissolution medium. Samples (2 m) were withdrawn at 15 min, 1, 2, and 4 h, centrifuged at 10,000 rpm for 5 min then analyzed using HPLC.

### Results

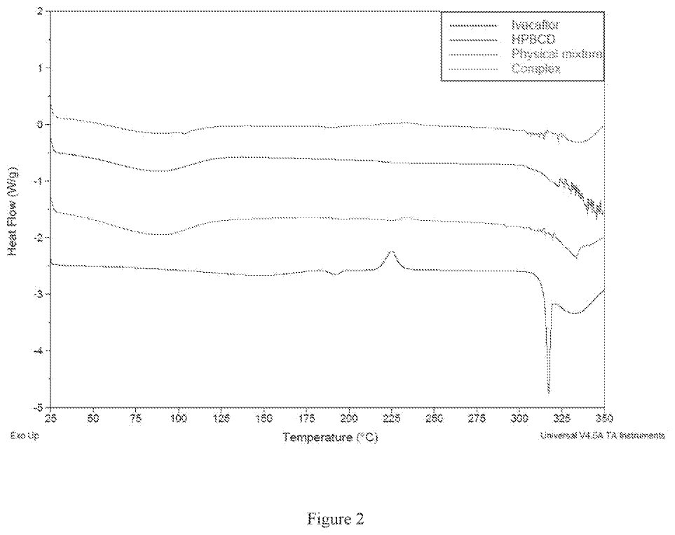

In contrast to the ivacaftor powder, the lyophilized powder obtained were highly dispersible in water. DSC profiles show that ivacaftor exhibits a strong endothermic peak at about 318° C., corresponding to the melting of ivacaftor. It also exhibits a small endothermic peak at 192° C., and another exothermic peak at 224° C. (FIG. 2) which are most likely corresponding to a polymorph of ivacaftor.

The characteristic peaks related to ivacaftor (in the region 1800-1200 cm⁻¹ characteristic to C═O ketone, C═C aromatic, and C—N amine, as well as that characteristic to N—H amine at 3300 cm⁻¹) were significantly abolished or completely disappeared in the complex DSC profile. In the physical mixture, the melting peak at 318° C. also disappeared, however, the exothermic peak was still visible even though it was shifted to about 228° C. This may indicate that ivacaftor molecules may be strictly included within the HPβCD molecules. This effect may occur but to a lesser extent in the case of physical mixture (FIG. 2).

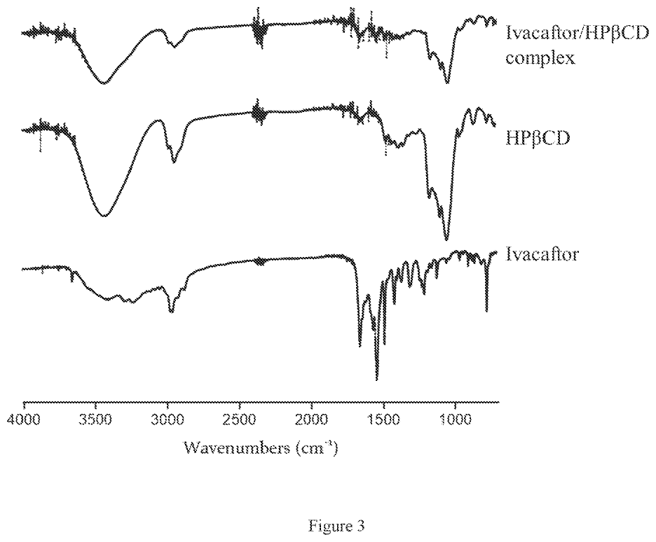

FTIR spectra showed that all signals related to ivacaftor were significantly abolished or completely disappeared, which may indicate that ivacaftor molecules are completely included within HPβCD molecules (FIG. 3).

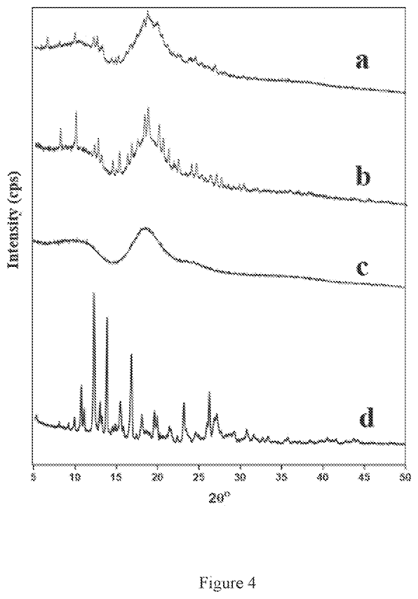

The diffraction pattern of ivacaftor showed sharp, intense peaks suggesting its crystalline nature. In contrast, due to the amorphous nature of HP-β-CD, its diffraction pattern showed two broad peaks with no crystalline peaks. The physical mixture diffraction pattern showed both, the two broad peaks corresponding to the amorphous nature of HP-β-CD, and the intense sharp crystalline peaks corresponding to ivacaftor. This indicates that there was no chemical association/interaction formed between both molecules in the physical mixture, since the pattern was a superposition of that of both molecules, suggesting that ivacaftor has retained its crystalline nature. On the other hand, the diffraction pattern of ivacaftor/HP-β-CD inclusion complex was analogous to that of HP-β-CD amorphous state with the crystalline peaks of ivacaftor almost disappeared, indicating that ivacaftor has lost most of its crystallinity as a result of its entrapment in the cavity of HP-β-CD (FIG. 4).

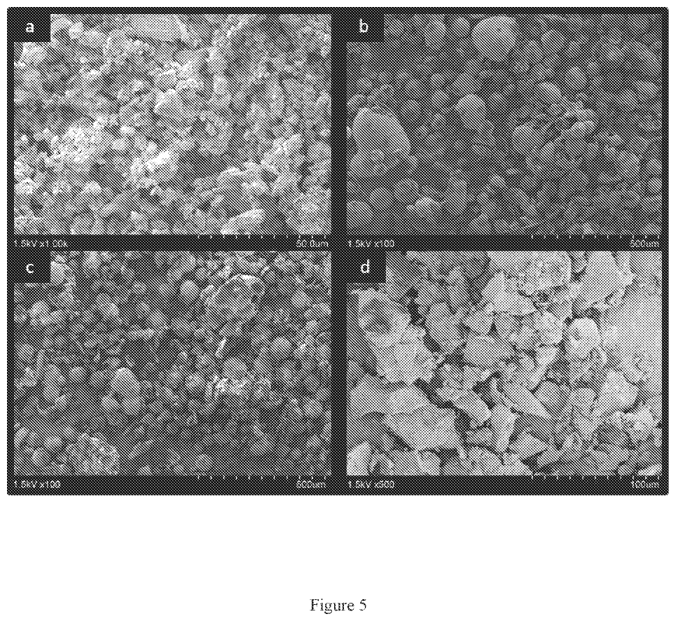

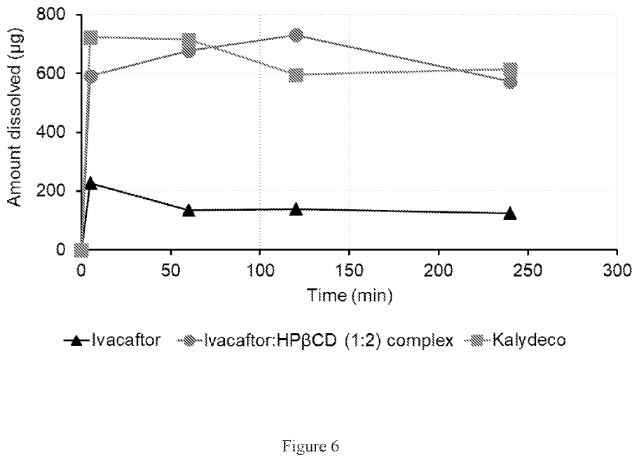

SEM photomicrographs of ivacaftor showed irregularly shaped crystalline structures with different sizes, HP-β-CD existed in its typical structure of amorphous, spherical particles with some cavity structures. The physical mixture SEM photomicrograph showed both the crystalline structures of ivacaftor and the spherical particles of HP-β-CD, indicating that there were no changes in the forms of both substances which means there was no interaction between them. However, in the ivacaflor/HP-β-CD inclusion complex, both ivacaftor and HP-β-CD original morphology have disappeared and instead exhibited a homogenous, organized blocky structure crystal various sizes. That significant change in particle morphology in the ivacaftor/HP-β-CD inclusion complex suggests the interaction between ivacaftor and HP-β-CD which indicates the formation of the inclusion complex. In the physical mixture sample, the original morphology of both ivacaftor and HP-β-CD didn't change, and the SEM photomicrographs simply showed a mixture of both structures, indicating there was no association between them. On the other hand, when ivacaftor and HP-β-CD where co-grinded in the presence of water-ethanol (1:1 v/v) solution and freeze dried, there was an interaction due to the formation of the inclusion complex, and as a result, ivacaftor didn't exist anymore in the crystalline state. In addition, the SEM photomicrographs (FIG. 5) are in line with the characterization results obtained by PXRD (FIG. 4) and DSC (FIG. 2) analysis which have showed that the crystalline characteristics of ivacaftor have disappeared upon the formation of the inclusion complex.

FIG. 6 shows that the amounts dissolved dissolution of ivacaftor from ivacaftor/HPβCD complex (1:2) was about 3-4 fold higher than those of ivacaftor alone, and the dissolution profile of ivacaftor/HPβCD complex (1:2) was comparable to that of Kalydeco® tablets.

### Conclusions

A complex between ivacaftor and HPβCD was successfully formed using the kneading method at a molar ratio of 1:2. The dissolution profile of the said complex is much faster than that of ivacaflor alone, and comparable to that of Kalydeco®.

### Example 3

To evaluate the pharmacokinetics (PK) following the intravenous (IV) injection of solubilized ivacaftor (aqueous solution in 10% polysorbate 80 v/v) in mice, the following experiments were conducted. The aims also included comparing the pharmacokinetic profiles of ivacaftor/HPβCD complex (1:2) and Kalydeco® tablets following the oral administration of both to mice.

### Methods

Female Balb-c mice (6-8 weeks old) were IV injected with an aqueous solution of ivacaftor containing 10% v/v polysorbate 80 at a dose of 0.1 mg/mouse in the tail vein. Mice were sacrificed at predetermined time points (n=3), and their plasma were collected and analyzed by HPLC.

In another experiment, female Balb-c mice were orally administered either crushed Kalydeco® tablets or ivacaftor/HPβCD complex (1:2) prepared by kneading method by oral gavage at a dose of 0.5 mg/mouse. Mice were sacrificed at predetermined time intervals (n=3), and their plasma were collected and analyzed by HPLC.

All PK compartmental analyses were performed using PK Solver Excel Add-in.

### Results

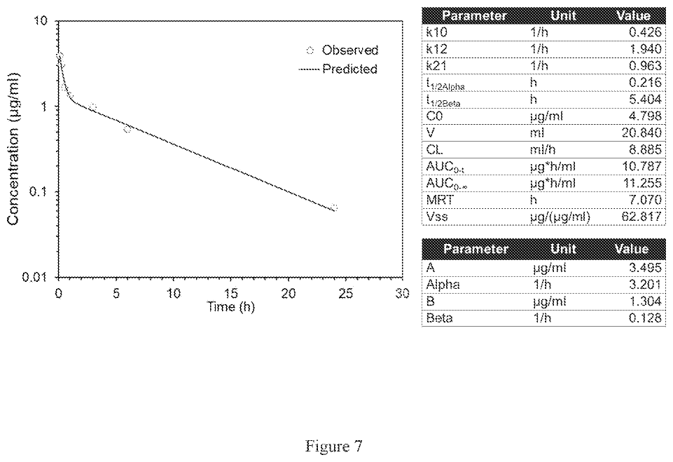

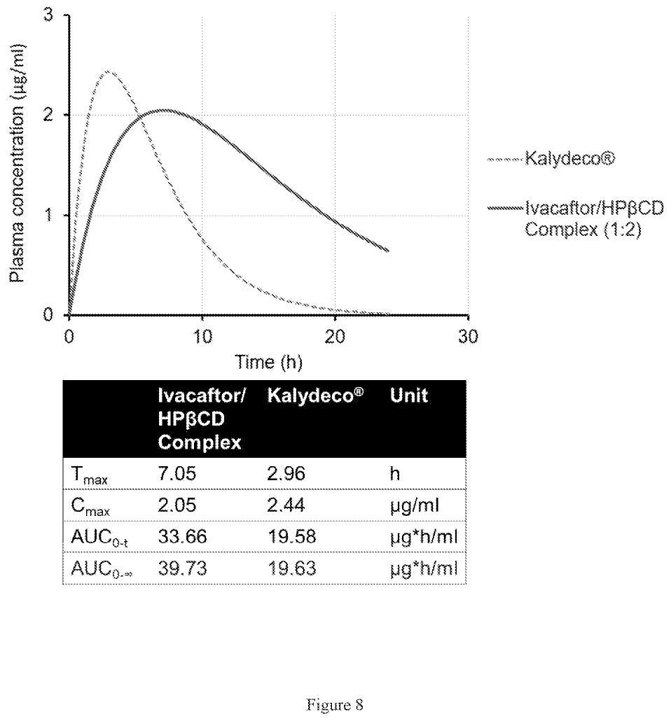

PK profile following IV injection of ivacaftor solution were found to follow 2-compartment model. Plasma levels and data can be found in FIG. 7.

In addition, PK profiles following oral administration of ivacaftor/HPβCD complex (1:2) or the commercial product showed that the complex provides higher bioavailability, as the area under the curve (AUC) of the complex is almost double that of Kalydeco®, and the Tₘₐₓ is about three times longer than that of Kalydeco® (FIG. 8). This indicates that the complex also provides sustained plasma levels, which may suggest a once-daily administration.

### Conclusion

The compositions comprising ivacaftor and HPβCD may provide sustained plasma levels, and higher oral bioavailability compared to the ivacaftor commercial product when given orally to mice.

### Example 4

The levels of ivacaftor in porcine plasma over 24 h after a single oral dose of ivacaftor/HPβCD complex (molar ratio of 1:2) and intramuscular (IM) administration of an aqueous solution comprising ivacaftor and polysorbate 80 were measured.

### Methods

10 kg pigs were given a single dose of ivacaftor either by IM injection of 10 mg/mL ivacaftor dissolved in polysorbate 80, or 1 dose of 20 mg/kg ivacaftor/HPβCD complex fed in milk. Blood draws were taken at baseline and 2, 4, 6, 8, 12, and 24 h after administration. Plasma ivacaftor levels were measured by Liquid chromatography-tandem mass (LC/MS), and concentrations were determined compared to standard curve, using lumacaftor as the internal standard.

### Results

Ivacaftor/polysorbate 80 solution given by IM injection showed a maximum plasma level of 11 μM at hour 6, and after 24 hours the plasma level remained about 80% of maximum. Ivacaftor/HPβCD given orally had a maximum plasma level of about 3 μM. This level was sustained through hour 8, and after 24 hours, remained close to 70% of the measured maximum.

### Conclusion

Preparations of ivacaftor/HPβCD complex given orally and also ivacaftor/polysorbate 80 given by IM injection are both capable of delivering sufficient levels of ivacaftor to correct G551D-CFTR function in pigs.

### Example 5

To check whether desirable levels of ivacaftor are found in the liver of porcine fetus following oral administration of the ivacaftor/HPβCD compositions, and intramuscular (IM) administration of an aqueous solution comprising ivacaftor and polysorbate 80, to pregnant sows, the following experiments were conducted.

### Methods

Compositions comprising ivacaftor and HPβCD at a molar ratio of 1:2 prepared using the kneading method described herein were given orally (3 doses at times 0, 12, 24, and 36 h) to a pregnant sow (weighing 151 kg) at a dose of 20 mg/kg at a gestational age of 33 days. After predetermined time points (0, 18, and 42 h following the administration of the first dose), plasma was collected and analyzed by LC/MS to determine ivacaftor levels. After 2 days, the sow was euthanized and the level of ivacaftor in the fetal liver was determined using LC/MS.

Also, an ivacaftor aqueous solution proposed herein (containing 20% v/v polysorbate 80) was injected IM at a dose of 20 mg/kg as 6 injections of 20 ml each in a pregnant sow (weighing 124 kg, n=1) at a gestational age of 34 days. After 1 day, the sow was euthanized and the level of ivacaftor in the fetal liver was determined using LC/MS.

### Results

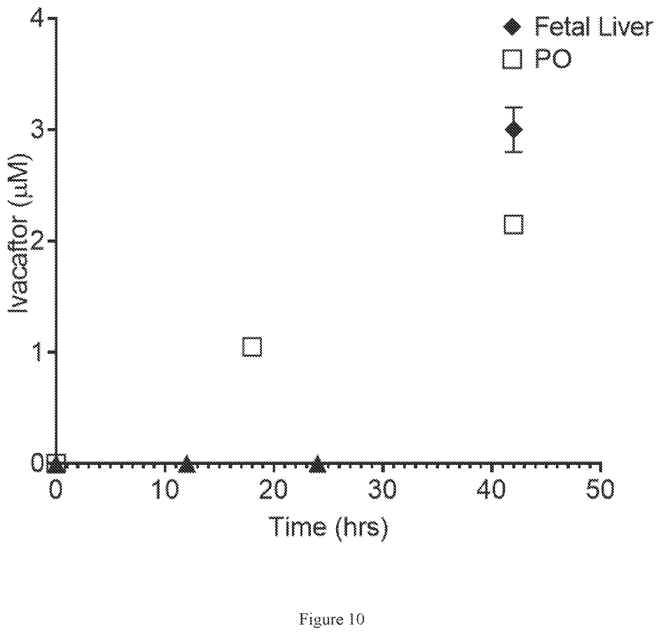

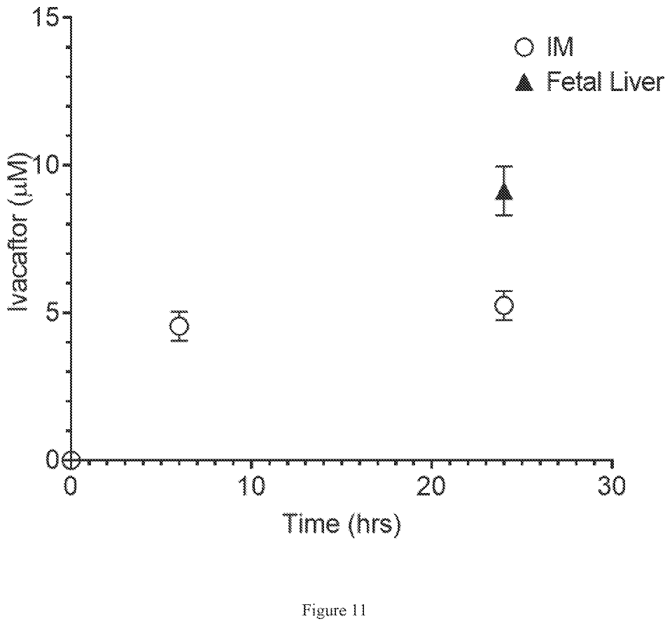

Plasma levels buildup and fetal hepatic levels of ivacaftor following multiple oral doses of the said composition or IM injection of the aqueous solutions containing ivacaftor and polysorbate 80 were all satisfactory and equal to or higher than what is described for treating humans and has been published for in utero correction in a ferret model of CF. (FIG. 10 and FIG. 11).

### Conclusion

The compositions comprising ivacaftor and HPβCD prepared using the kneading method given orally (ivacaftor/HPβCD complex at a molar ratio of 1:2) and also the proposed aqueous solutions of ivacaftor given by IM injection are both capable of delivering sufficient levels of ivacaftor to fetal tissues.

### Example 6

To determine if in utero exposure to ivacaftor could correct the lethal cystic fibrosis phenotype present at birth, allowing animal survival for longitudinal study of disease, the following experiments were run.

### Methods

The composition comprising ivacaftor and HPβCD at a molar ratio of 1:2 prepared using kneading method was given orally to two pregnant sows (approximately 145 kg, 35 days gestation) with G551D-CFTR at a dose of 5 mg/kg bid for a total of 85 days.

### Results

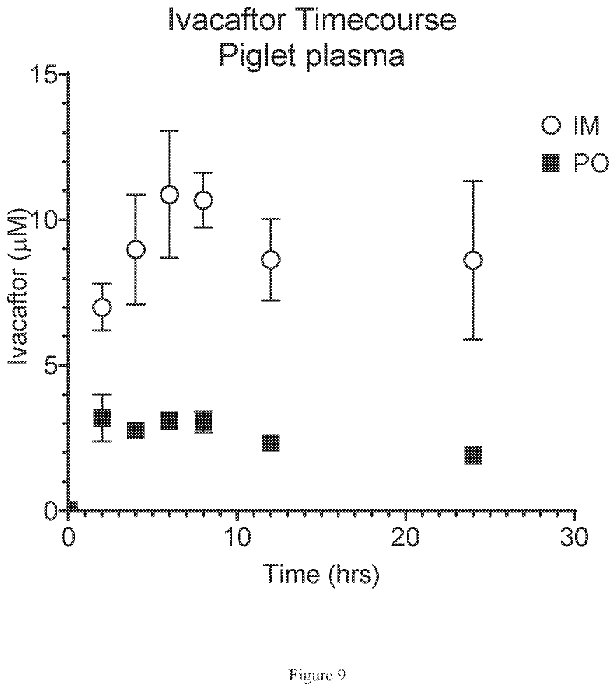

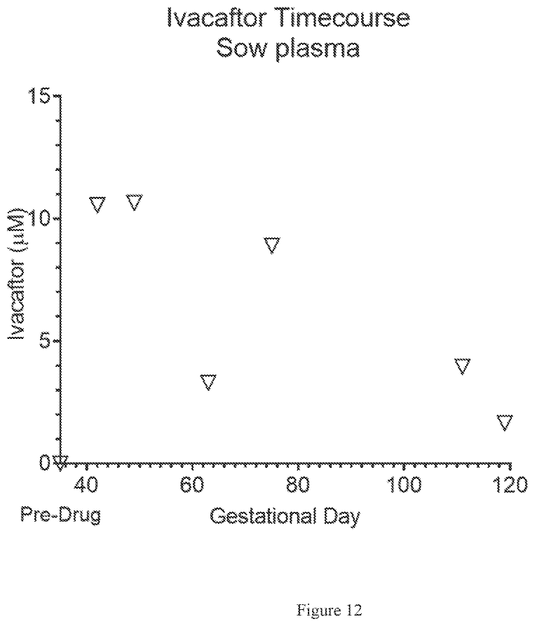

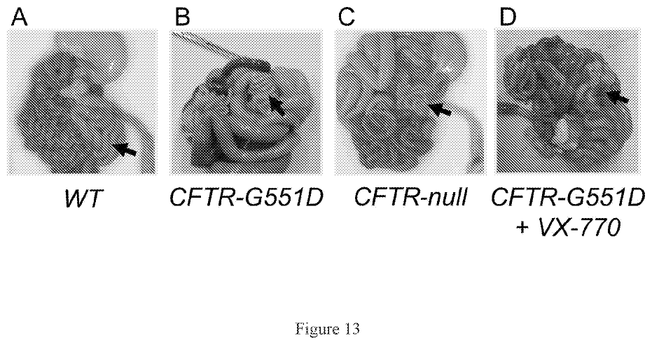

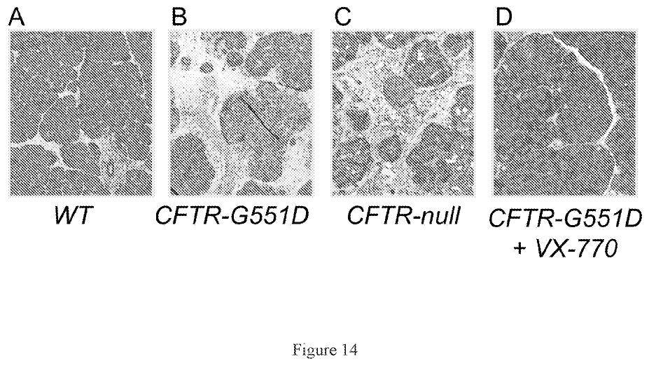

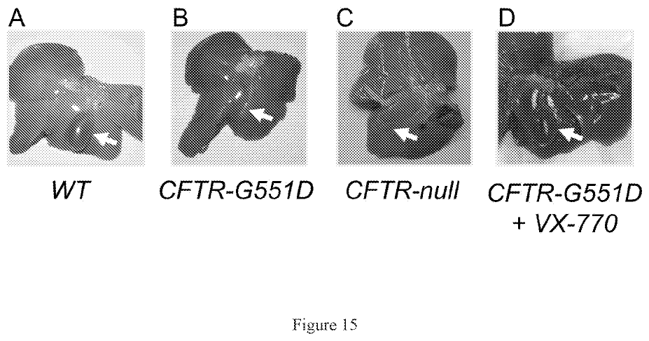

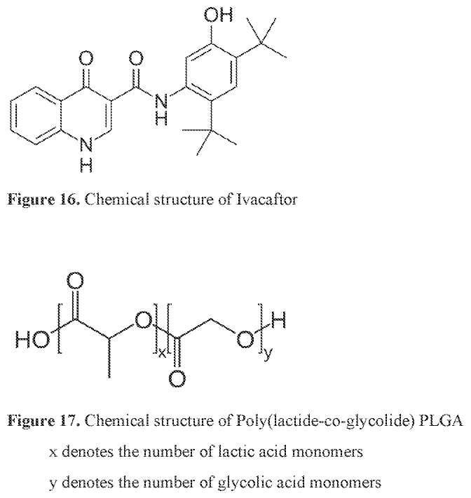

Plasma levels of ivacaftor remained at or above what has been published for humans with CF and a ferret model of CF until 114 days of gestation. After 114 days, the levels fell slightly, likely due to reduced animal intake. One piglet from the resulting litter was euthanized soon after birth and was found to have a relatively normal-appearing intestine, pancreas and gallbladder (FIG. 9-12).

### Conclusion

Administering ivacaftor/HCBD complex to pregnant sows provided fetal levels of ivacaftor that corrected lethal intestinal phenotype of CF piglets. Importantly, intestinal rescue provided by ivacaftor allowed piglets from this litter to be aged and studied after elective euthanasia. Previously, this could only be done by risky surgical correction on the day of birth, or further genetic manipulation.

### Example 7

Ivacaftor (IVA) is a drug used for the treatment of cystic fibrosis (CF) among a class of drugs called CFTR gene modulators. It is available in the market since 2012 in the form of oral tablets under the brand name Kalydeco that is developed by Vertex® pharmaceuticals. To reach therapeutic levels of ivacaftor in the blood, these tablets (150 mg each) must be administered by the patients twice daily. This may negatively affect the lifestyle of the patients, resulting in incompliance and ultimately treatment failure.

### Materials and Methods

### Materials

All chemicals and reagents used were of analytical or HPLC grade. Ivacaftor was purchased from Vertex pharmaceuticals (Boston, MA). Poly lactide-co-glycolide (PLGA, Resomer RG 503 H, m.wt=24,000-38,000, viscosity=0.32-0.44 dL/g in chloroform and Resomer RG 502, m.wt=7,000-17,000, viscosity=0.16-0.24 dL/g in chloroform) were purchased from Evonik (Parsippany, NJ). Poly vinyl alcohol (PVA, Mowiol 8-88, MW 67,000), screw-capped dialysis tube (Spectra/Por™ Float-A-Lyzer™ G2, MWCO 50 kDa), and phosphate buffered saline (PBS) tablets were purchased from Sigma Aldrich (St. Louis, MO). Tween-80, methylene chloride (dichloromethane, DCM) and acetonitrile were purchased from Fisher Chemicals (Waltham, MA).

### Preparation of PLGA Microparticles

IVA-loaded poly(lactide-co-glycolide) (PLGA) microparticles were prepared using an oil-in-water single emulsion solvent evaporation technique as previously described (Naguib et al., 2020; Khaled et al., 2010). Briefly, 50 mg of the PLGA polymer and 5 or 10 mg IVA were dissolved in 1.5 mL dichloromethane (DCM) (organic phase). The organic solution was then added to 30 mL (1, or 1.5% w/v) polyvinyl alcohol (PVA) solution and the mixture was immediately emulsified for 1 min at (6500 or 13500 rpm) at room temperature using an overhead homogenizer (Ultra-turrax T25 basic, Ika Works, Inc., Wilmington, NC). The emulsion was then stirred using a magnetic digital stirrer at 25° C. set at speed 300 rpm for 2 h to evaporate DCM. To collect the microparticles, the suspension was centrifuged at 1000×g for 5 min (Eppendorf centrifuge 5864 R, Eppendorf North America, Hauppauge, NY). The collected microparticles were resuspended in 30 mL nanopure water (Barnstead Thermolyne Nanopure water purification system, Thermo Fisher, Waltham, MA), washed twice at the same centrifugation conditions to remove any remaining PVA or unencapsulated drug. Finally, the microparticles were resuspended in 1 mL nanopure water, frozen at −80° C. and lyophilized overnight at 0.045 mbar and a collector temperature of −105° C. (Labconco Free zone 4.5 L-105° C. Labconco, Kansas City, MO). After 24 h of lyophilization, particles were collected and stored at −20° C. until use.

### High Performance Liquid Chromatography (HPLC-UV) and Liquid Chromatography Tandem-Mass Spectrometry (LC-MS/MS)

### HPLC-UV

An HPLC-UV method for the quantification of IVA has been developed using an Agilent workstation (Agilent Infinity 1100, Santa Clara, CA) coupled with an Agilent diode array detector (DAD). A reversed phase Waters® Symmetry Cis column (5 μm pore size, 4.6 mm i.d.×150 mm) (Waters, Milford, MA, USA) was used for analysis. The mobile phase consisted of a mixture of Acetonitrile: water (60:40) with 0.1% v/v trifluoroacetic acid in an isocratic elution mode. The flow rate and the injection volume were set at 1 m/min and 50 μL, respectively, at room temperature. Detection wavelength was set to 309 nm. Stock solution of ivacaftor was prepared in methanol at a concentration of 1 mg/ml. To construct a calibration curve, ivacaftor stock solution was diluted to 6 calibration standards in the range of 0.1-50 μg/mL using Acetonitrile:water (50:50 v/v) and injected into the HPLC-UV, data was collected and a linear regression equation was fit into the calibration standards

### LC-MS/MS

The chromatographic analysis and mass spectrometric detection was carried out using a Waters® Acquity H-class ultra-performance liquid chromatography (UPLC) system coupled with a Waters® XEVO TQ-S cronos triple quadrupole mass spectrometer (Waters corporation, Millford, MA) operating in positive electrospray (ESI) and multiple reaction monitoring (MRM) mode. Chromatographic separation was performed on an Agilent® RRHD Eclipse Plus Ce column (2.1 i.d.×100 mm, 1.8 um pore size) (Agilent technologies, Santa Clara, CA). The mobile phase consisted of a mixture of (A) water with 0.1% trifluoroacetic acid v/v and (B) acetonitrile. The initial gradient conditions were 60% (B) for 6 min, gradually increased to 95% over 1 min and maintained at this concentration for an additional 4 min then switched back to 60% for 6 min to re-equilibrate the column. Total run time was 17 min, the flow rate was set at 0.2 mL/min with an injection volume of 20 μL/sample.

The mass spectrometric detection of IVA and the internal standard (IS) LUM (VX-809) was conducted in positive ESI via MRM mode. The optimum MRM transitions were m/z 393.18 A 172.07 and 453.02 A 131.04 for quantification of IVA and LUM, respectively and data was acquired between 3.5 and 8 min of the run time. Fragments were induced using a collision energy (CE) of 28 V for IVA and 40 V for LUM. Additional ESI source parameters included a source temperature of 150° C., nitrogen desolvation and cone gas flow of 800 and 50 L/h, respectively. A capillary and cone voltage of 1 kV and 16 V, respectively.

### Standard Solution and Calibration Curve

Stock solution of IVA was prepared in methanol while that of the internal standard LUM was prepared in acetonitrile, both at a concentration of 1 mg/mL. IVA stock solution was diluted into the working solutions range of (0.01-25 μg/mL) using methanol and LUM was diluted to a working concentration of 2.5 μg/mL. Stock solutions of IVA and LUM were stored in −80° C., working solutions of both IVA and LUM were prepared freshly on the day of analysis. To construct the calibration curve, 100 μL of blank mouse plasma was spiked with 10 μL of each IVA working solution (range: 0.01-25 μg/mL for a final concentration range of 0.001-2.5 μg/mL). IVA was extracted from blank plasma spiked with calibration standards and mice plasma study samples as describe in the sample preparation below.

### Characterization of the Microparticles

### Microparticles Surface Morphology

The size and surface morphology of the prepared microparticles formulations were investigated using a Hitachi S-4800 scanning electron microscope (SEM) (Hitachi High Technologies, Ontario, Canada) as described previously. The lyophilized microparticles were spread onto a carbon double-adhesive tape that is mounted on aluminum stub, and the surface was coated with gold and palladium (to make the samples electrically conductive) using an argon beam K550 sputter coater (Emitech Ltd., Kent, U.K). SEM photomicrographs of the microparticles were captured at 1.5 kV accelerating voltage. A minimum of 100 particles in the captured SEM images were analyzed to obtain average particle size and standard deviation using ImageJ software (NIH, Bethesda, MA). The obtained data was plotted in histograms using GraphPad Prism software (GraphPad, San Diego, CA).

### Microparticles Size Analysis

To get an accurate estimate of the particles size distribution, particles size data was further analyzed to obtain D₁₀, D₅₀, and D₉₀ values which represent the sizes below which 10, 50, and 90% of the microparticles fell. These values were used to calculate the span value which gives an estimate to the dispersibility and uni

uniformity of the microparticles. Span value was calculated using the following equation (1):

$$Span = \frac{( {D}_{90} - {D}_{10} )}{{D}_{50}}$$

### Determination of Microparticles Drug Loading and Encapsulation Efficiency

The prepared microparticle formulations were dissolved in acetonitrile at 1 mg/mL by sonication for 10 min until clear solution was obtained. Then 100 μL of this solution was transferred to 900 μL (500 μL acetonitrile+400 μL nanopure water) to make a 10-fold dilution. The solution was vortexed and centrifuged (14,000×g for 5 min) and the supernatant was analyzed using HPLC-UV as described below

The microparticles' drug content was calculated using equation (2):

$$Drug Loading ( \frac{ug}{mg} ) = \frac{IVA concentration ( \frac{ug}{mL} ) \times volume ( mL )}{Particles weight ( mg )} \qquad ( 2 )$$

The particles yield percentage was calculated using equation (3):

$$Yield ( \% ) = \frac{Weight of lyophilized particles ( mg )}{Weight of starting materials ( mg )} \times 100 \qquad ( 3 )$$

Finally, encapsulation efficiency percentage (EE %) was calculated using equation (4)

$$Encapsulation efficency ( \% ) = \frac{Drug loading ( \frac{ug}{mg} ) \times yield of particles ( mg )}{Initial amount of drug added ( ug )} \times 100 \qquad ( 4 )$$

### In Vitro Drug Release Kinetics

An accurate amount of IVA-loaded PLGA MPs equivalent to 270 μg IVA (based on the drug loading μg/mg) was weighed out and suspended in 1 mL 1×DPBS (Dulbecco's phosphate-buffered saline, Life Science, Waltham, MA). The 1 mL particles suspension was transferred to a 1 mL screw-capped dialysis tube (Spectra/Por™ Float-A-Lyzer™ G2 MWCO 50 kDa, Sigma-Aldrich) and the tubes were immersed in 12 mL 0.4% v/v solution of polysorbate 80 in 1×DPBS and transferred to an orbital shaker (New Brunswick Scientific. Edison, NJ) set at 300 rpm and 37° C. Ivacaftor's solubility in the release medium was 271.5 t 3.055 μg/ml at 37° C. At pre-determined time points; 1, 3, 6, 24, 48, 72 h and 7, 10, 14, 17, 21, 24, and 30 days, 1 mL was sampled for analysis, and the remaining release medium was discarded and replaced with 12 mL fresh release medium. Ivacaftor concentration in the collected samples was measured using HPLC-UV as described below. Each formulation was tested in 3 replicates, and the data was represented as average cumulative release (%)±standard deviation (SD).

### In Vivo Pharmacokinetics Study

### Study Design

Pharmacokinetics of soluble IVA and in vivo release of IVA-loaded PLGA microparticles (Formulation 1) studies were performed on a total of 51 male BALB/CJ mice (8 weeks old, weighing 20-25 g, Jackson labs, Bar Harbor, ME). Mice were kept at the university of Iowa animal care facility under controlled temperature (23 t 2° C.) and were exposed to 12 h light and dark cycles. To study pharmacokinetics of soluble IVA, twenty-one mice were injected intravenously (via tail vein injection) with a solution of IVA dissolved in 10% polysorbate 80 in sterile 1×DPBS at a concentration of 1 mg/mL (total volume 100 μL for a dose of 5 mg/kg). Three mice were sacrificed at pre-determined time points: 5, 15, 30, 60, 180, 360, 1440 min by injecting 100 μL Ketamine/Xylazine (87.5/12.5 mg/kg) solution intra-peritoneally, followed by cervical dislocation. Blood samples were collected immediately via cardiac puncture into 1.5 mL Eppendorf® tubes containing 10 μL sodium heparin (1000 USP units/mL) to prevent coagulation. Plasma was separated from the supernatant after centrifugation of the blood samples at 14,000×g for 10 minutes and frozen at −80° C. until analysis by LC-MS/MS to determine the IVA plasma levels.

To study the pharmacokinetics and in vivo release of IVA-loaded PLGA microparticles (Formulation 1), the microparticles were suspended in 1×DPBS and thirty mice were injected subcutaneously (SC) at a dose of 1 mg IVA (50 mg/kg) in 0.3 mL/mouse. At each time point: 1, 4, 24 h; 7, 10, 14, 21, 24, and 28 days, three mice were sacrificed, and blood samples were collected, processed and stored, as described above, until analysis by LC-MS/MS.

### Plasma Samples Preparation

Analysis of plasma samples to determine IVA concentration was done at room temperature. Acetonitrile protein-precipitation technique was carried out to extract IVA from plasma samples. Briefly, plasma samples were thawed and aliquots of 100 μL were transferred to 1.5 mL Eppendorf® tubes and spiked with 10 μL of the internal standard working solution (LUM 2.5 μg/mL for a final concentration of 0.25 μg/mL). Then 1 mL of cold acetonitrile was added, samples were vortexed for 1 min and transferred to ice bucket for 10 min to allow plasma proteins precipitation. Samples were then centrifuged at 4.000×g for 10 min at 4° C., and the supernatant was transferred to glass tubes and evaporated under a light stream of nitrogen. The residue was reconstituted in 100 μL acetonitrile:Water (50:50 v/v), vortexed and centrifuged for 5 min at 14,000×g and 20 μL of the supernatant was injected to the LC-MS/MS for quantification.

### Statistical Analysis

All experiments were repeated 3 times. Microsoft Excel (2010) was used to analyze the in vitro release data, calculate % cumulative release and perform span analysis for microparticles sizes.

GraphPad Prism software (GraphPad, San Diego, CA) was used for creating all figures and graphs presented in this work. Non-compartmental pharmacokinetics (NCA) analysis was performed using PkSolver (an Excel Ad-in) (Yang et al., 2000).

### Results and Discussion

### Development of the Microparticles

IVA-loaded microparticles were prepared using a single-emulsion (oil in water) solvent evaporation method, owing to the hydrophobic nature of ivacaftor (log P=3.13). Dichloromethane (DCM) was chosen as solvent due to its ability to solubilize all kinds of PLGA regardless of the lactide:glycolide monomer ratio, in addition to its low boiling point (39.8° C.) which allows fast evaporation (Park et al., 2020; Liu et al., 2003). Polyvinyl alcohol was used as an emulsifier due to its superior chemical and physical properties such as being non-toxic, biodegradable, biocompatible and stable towards temperature variation (Gaaz et al., 2015). To obtain an IVA-loaded microparticle formulation with the best formulation characteristics (particle size, drug loading, encapsulation efficiency, drug release kinetics), the effect of varying four formulation parameters on the produced microparticle formulation characteristics was studies. The formulation parameters varied are presented in Table 1. The effect of varying the formulation parameters on the IVA-loaded microparticles formulations characteristics are shown in Table 2.

TABLE 1
Formulation parameters of the six ivacaftor-
loaded PLGA microparticle formulations.

| | IVA | Polymer (PLGA) | | Emulsifier | | Emulsification | | Varied |
|---|---|---|---|---|---|---|---|---|
| | Amount | | Amount | | Conc. | Speed | Time | formulation |
| F# | (mg) | Type | (mg) | Type | (%) | (rpm) | (min) | parameter |
| 1 | 5 | Resomer ® | 50 | PVA | 1 | 6500 | 1 | Drug:polymer |
| | | RG 503 H | | | | | | ratio |
| 2 | 10 | Resomer ® | 50 | PVA | 1 | 6500 | 1 | |
| | | RG 503 H | | | | | | |
| 3 | 5 | Resomer ® | 50 | PVA | 1.5 | 6500 | 1 | Emulsifier |
| | | RG 503 H | | | | | | concentration |
| 4 | 5 | Resomer ® | 50 | PVA | 1 | 9500 | 1 | Emulsification |
| | | RG 503 H | | | | | | speed |
| 5 | 5 | Resomer ® | 50 | PVA | 1 | 13500 | 1 | |
| | | RG 503 H | | | | | | |
| 6 | 5 | Resomer ® | 50 | PVA | 1 | 6500 | 1 | Polymer |
| | | RG 502 H | | | | | | molecular |
| | | | | | | | | weight |

Resomer RG 503 H: PLGA (50:50) Mw (24,000-38,000) acid terminated
Resomer RG 502 H: PLGA (50:50) Mw (7,000-17,000) acid terminated
PVA: Polyvinyl alcohol

| | | Polymer | | Polymer | Ivacaftor | |
|---|---|---|---|---|---|---|
| F# | Polymer | viscosity (IV)* | | amount (g) | (g) | Loading % |
| F1A | IPLGA 75:25 | 0.5-0.75 | dl/g | 4.5 | 1.5 | 25% |
| F2A | 2PLGA 75:25 | 0.15-0.25 | dl/g | 4.5 | 1.5 | 25% |
| F3A | 3PLGA 50:50 | 0.5-0.65 | dl/g | 4.5 | 1.5 | 25% |
| F4A | 4PLGA 50:50 | 0.15-0.25 | dl/g | 4.5 | 1.5 | 25% |

*reported inherent viscosity is in chloroform (250, 1.0 g/dL)
Links for polymers
¹https://www.polysciences.com/default/polydl-lactide-co-glycolide-7525
²https://www.polysciences.com/default/26268
³https://www.polysciences.com/default/polydl-lactideglycolide-9023
⁴https://www.polysciences.com/default/50-50-poly-d-l-lactide-co-glycolide-acid-terminated-iv-0-2-dl-g-mw-15000

TABLE 2
Characteristics of the six ivacaftor-loaded PLGA microparticle formulations

| | Drug | Encapsulation | | | | | Average | |
|---|---|---|---|---|---|---|---|---|
| | loading | efficiency (EE) | Yield | D₁₀ | D₅₀ | D₉₀ | diameter | Span |
| F# | % (w/w + SD)¹ | % (w/w ± SD)¹ | % (w/w ± SD)¹ | (μm) | (μm) | (μm) | (μm ± SD)¹ | value |
| 1 | 8.25 ± | 90.7 ± | 48.5 ± | 5.26 | 6.83 | 8.3 | 6.83 ± | 0.28 |
| | 3.73 | 41.1 | 8.96 | | | | 1.18 | |
| 2 | 10.3 ± | 56.7 ± | 58.3 ± | 5.45 | 6.71 | 8.61 | 6.93 ± | 0.35 |
| | 6.6 | 36.3 | 2.21 | | | | 1.31 | |
| 3 | 4.48 ± | 49.3 ± | 61.4 ± | 5.38 | 6.36 | 7.38 | 6.38 ± | 0.19 |
| | 0.7 | 7.71 | 10.5 | | | | 0.77 | |
| 4 | 3.91 ± | 43 ± | 70.3 ± | 2.6 | 3.28 | 3.99 | 3.32 ± | 0.26 |
| | 0.37 | 4.09 | 9.15 | | | | 0.58 | |
| 5 | 2.59 ± | 26.6 ± | 63.6 ± | 1.47 | 1.9 | 2.43 | 1.91 ± | 0.36 |
| | 0.88 | 9.71 | 15.3 | | | | 0.41 | |
| 6 | 5.84 ± | 100 ± | 64.1 ± | 4.26 | 5.51 | 7.19 | 5.64 ± | 0.39 |
| | 3.55 | 39.1 | 5.9 | | | | 1.12 | |

¹Mean ± standard deviation (SD, n = 3)

### Microparticle Size and Surface Morphology

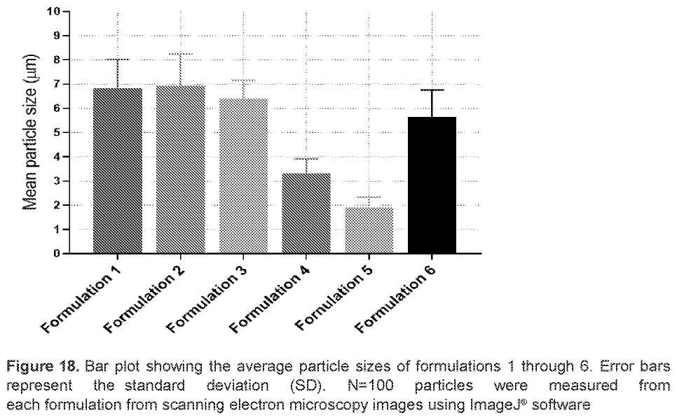

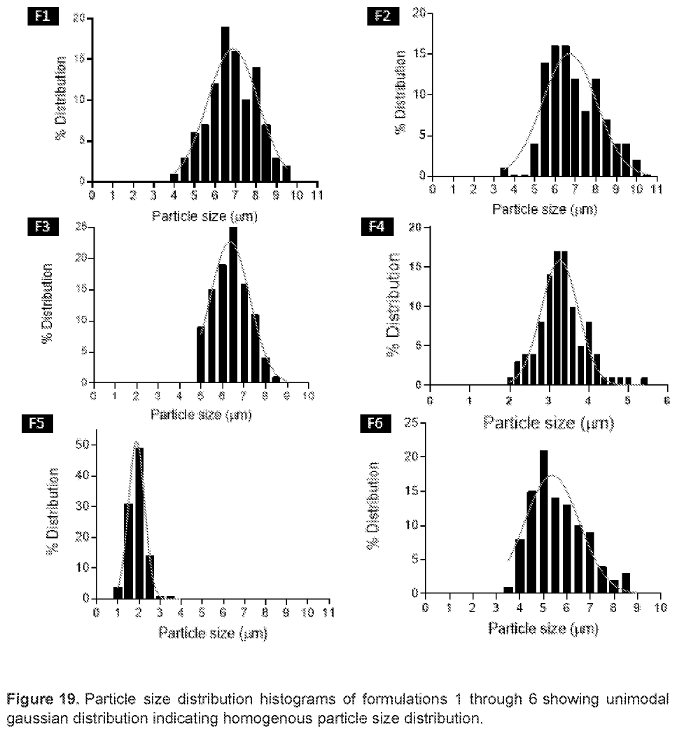

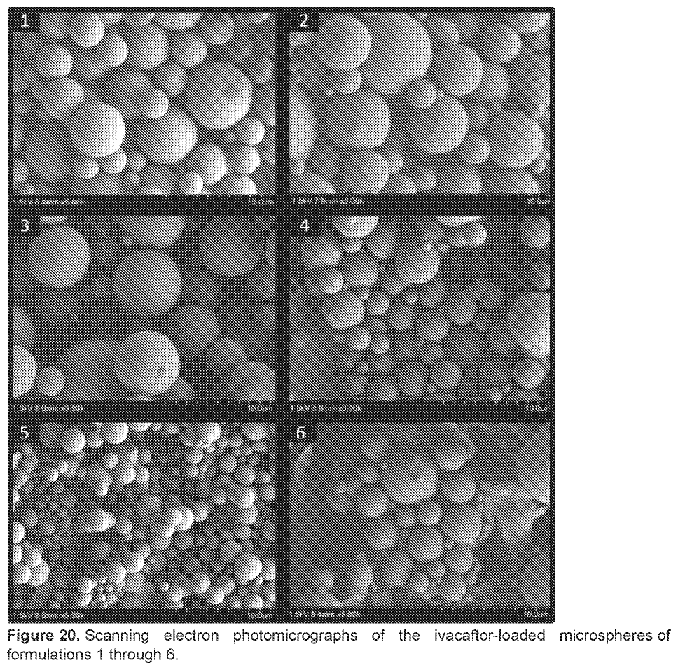

The produced IVA-loaded microparticles had an average diameter that ranged from 1.91-6.93 μm with narrow size distributions shown by span values ranging from 0.19-0.39. The average particle size of each formulation as measured by ImageJ software utilizing 100 particles is represented in FIG. 18. Particle size distribution histograms of all formulations are presented in FIG. 19, and was generally unimodal indicating a homogenous distribution of the particle sizes. SEM photomicrographs (FIG. 20) revealed particles with mostly smooth, non-porous, spherical shape with no unencapsulated drug crystals. The effect of increasing the drug:polymer ratio from 10% (formulation 1) to 20% (formulation 2) was reflected in a bumpy surface in formulation 2 as compared to formulation 1 which had a smooth surface. This could be due to the presence of more drug molecules encapsulated in formulation 2 in crystal form rather than molecular dispersions due to the hydrophobicity of ivacaftor (Naguib et al., 2009; Mao et al., 2008). Changing the PVA surfactant concentration had a clear effect on particle size. Surfactants play a role in maintaining the interfacial tension between the aqueous and organic phase to maintain the stability of the emulsion (Park et al., 2020). Increasing the surfactant concentration from 1% in F1 to 1.5% in F3 has produced smaller particle size due to the reduction in interfacial tension and improvement of emulsion stability. A 1% PVA surfactant concentration (formulation 1) was sufficient to maintain the stability of the emulsion and produce particles with the desired the size. Changing the stirring speed resulted in a change in the produced microparticles size. Higher stirring speeds in formulations 4 and 5 resulted in smaller particle sizes with an average diameter of 3.32 and 1.91 μm, respectively as compared to formulation 1 (with lower stirring speed) that had an average diameter of 6.83 μm. This is due to the breakdown of the oil phase into smaller microdroplets due to the higher shearing forces provided by the higher stirring speeds (Yang et al. 2000). Finally, decreasing the polymer molecular weight in formulation 6 (RG502H) has resulted in a smaller particle size with an average diameter of 5.64 μm when compared to formulation 1 where a higher molecular weight polymer (RG503H) was used. This can be explained by the higher viscosity of the oil phase when a higher molecular weight polymer is used which resists the shearing force during the emulsification process resulting in a larger average particle size (Xue et al. 2004).

### In Vitro Drug Release Kinetics, Drug Loading and Encapsulation Efficiency

PLGA is a bulk (homogenous) eroding biopolymer where drug release from the microspheres is driven by a combined diffusion-erosion mechanism (Han et al., 2016). Drug release profiles of PLGA microspheres typically follow a bi/tri-phasic pattern. At first, an initial rapid burst release is observed due to the diffusion/dissolution of the drug near or at surface of the microspheres. This is followed by a slow-release lag-phase which occurs due to the time needed for build-up of acidic degradation moieties, resulting from the hydrolytic cleavage of the PLGA ester bonds, to

reach sufficient concentrations before the bulk-erosion phase of the polymer is initiated. PLGA physicochemical properties (mainly Lactide:glycolide ratio and molecular weight) and physicochemical properties of the encapsulated drug (mainly hydrophilicity and molecular weight) all influence the extent of drug release at each phase and the lag time between the two phases, where in some formulations, the lag phase is too brief that the release profile appears biphasic (Han et al., 2016; Park et al. 2019; Khaled et al., 2010; Naguib et al., 2009). The effect of varying different formulation parameters (drug:polymer ratio, surfactant concentration, emulsification speed, PLGA molecular weight) on the drug loading, encapsulation efficiency and release of ivacaftor from the PLGA microspheres has been studied in this work to produce ivacaftor-loaded microparticle formulation with optimized characteristics.

### Effect of Drug Polymer Ratio (D:P)

The effect of increasing the initial amount of added drug 10 vs 20% (1:10 vs 2:10, D:P) on the drug loading (DL), encapsulation efficiency (EE %) and release kinetics of formulations 1 (F1) and 2 (F2), respectively, while keeping other formulation parameters constant, was studied. F2, with a higher initial added drug amount (20%) had a higher average drug loading (±SD) of 10.3% (±6.6) vs 8.25% (±3.73) for F1 (Table 2). This is expected, since ivacaftor is a lipophilic drug, when more drug is initially added to the microparticle formulation, more drug stays in the inner oil phase rather than escaping to the external aqueous phase which results in higher drug loading. The higher drug loading in F2 resulted in a weaker average initial burst release (±SD) of 14.14% (±8.77) vs 23.73% (±5.6) in F1 after 3 days, and an overall slower % cumulative drug release (FIG. 21A). This is again due to the hydrophobic nature of ivacaftor, where it has been reported that hydrophobic drugs tend to form crystals in the microparticles as the drug loading increases (Naguib et al., 2009; Mao et al., 2008; Chen et al., 2004). Since F2 has relatively higher drug loading than F1 (10.3 vs 8.25%), more of the encapsulated drug in F2 would exist in crystal form rather than being molecularly dispersed. These drug crystals require an extra dissolution step which causes the release to be slower in F2. Finally, in contrast to drug loading, F2 had a lower average encapsulation efficiency (±SD) of 56.7% (±36.3) in comparison to F1 with 90.7% (±41.1). This can happen when the oil phase has reached its maximum saturation with the drug, so adding more drug would result in a lower encapsulation efficiency (Cheng et al., 1998).

### Effect of Surfactant Concentration

To understand the effect of changing the surfactant concentration on the microparticle formulation characteristics, in formulation 3 (F3), a higher surfactant concentration (1.5% PVA) than that used in F1 (1%) was used. As described earlier, increasing the surfactant concentration has led to a decrease in particle size, this is because increasing the surfactant concentration improves the emulsion stability and reduces the interfacial tension between the aqueous and oily phase resulting in smaller particle sizes. FIG. 6b shows the release profiles of F1 and F3. Since F3 (with smaller particle size) has a larger specific area per unit volume, it had a stronger initial burst release and an overall faster cumulative release rate than of F1. After 3 days, 70% (±14.3) of ivacaftor has been released from F3 vs only 23.7% (±5.6) from F1, and by 6 weeks, F1 has released 66.4% (±16.89) of the loaded ivacaftor vs 86.1% (±14.7) released from F3. Increasing surfactant concentration also had an impact on lowering both DL and EE. F3 had an average DL (±SD) and EE (±SD) of 4.48% (±0.7) and 49.3% (±7.71) vs 8.25% (±3.73) and 90.7% (±41.1) for F1, respectively. The decrease in DL and EE upon increasing the surfactant concentration could be due to the smaller particle size of F3, in addition to ivacaftor being less likely to stay in the formulation due to the high surfactant concentration in the surrounding aqueous medium during the solvent evaporation step.

### Effect of Stirring Speed

The effect of stirring speed on the formulation characteristics, by increasing the speed from 6500 rpm (F1) to 9500 (F4) and 13500 (F5), was examined. Higher emulsification speed generates higher shear force which breaks down the oil phase into smaller droplets. When the small oil droplets evaporate, they result in small particle sizes. The data has shown that trend, where, F1 (with the lowest emulsification speed) had an average particle size diameter (±SD) of 6.83 μm (±1.18) compared to 3.32 μm (±0.58) and 1.91 μm (±0.41) for formulations 4 and 5, respectively. Smaller particle sizes may have lower drug loading and encapsulation efficiency due to the smaller inside diameter of the particles where the drug can be loaded (Yang et al. 2000). Formulation 1 had average drug loading (±SD) and encapsulation efficiency (*SD) of 8.25% (±3.73) and 90.7% (±41.1) compared to 3.91% (±0.37) and 43% (±4.09) for formulation 4, 2.59% (±0.88) and 26.6% (±9.71) for formulation 5, respectively (Table 2). Regarding the in vitro release kinetics, in smaller particles, the encapsulated drug needs to travel a shorter distance to the outside of the particles. In addition, the total surface area of particles exposed to the release medium is large. Both factors result in an overall faster cumulative release rates and a stronger initial burst release with particles of small size. FIG. 21C shows the in vitro cumulative release profiles of formulations 1, 4, and 5. After 3 days, formulation 1 have released (±SD) 23.7% (±0.44) vs 29.13% (±22.41) from formulation 4 and 46.63% (±11.53) from formulation 5. By 10 days, 35.24% (±10.4) of ivacaftor have been released from formulation 1 vs 54.1% (±15.3) and 55.8% (±9.4) released from formulations 4 and 5, respectively. And finally at the end of the release study (6 weeks), 66.46% (±16.8) of ivacaftor have been released from formulation 1 vs 66.9% (±16.5) and 71.1% (8.1) released from formulations 4 and 5, respectively.

### Effect of Polymer Molecular Weight

To study the effect of changing the polymer molecular weight on the DL, EE % and release kinetics of the microparticles, in formulation 6 (F6), a polymer of lower molecular weight (mol.wt.) and inherent viscosity than that used in formulation 1 (F1) was tested. The polymers used in F1 and F6 were an RG503H and RG502H with mol.wt. ranges of 24,000-38,000 and 7,000-17,000 and inherent viscosities of 0.32-0.44 and 0.16-0.24 dL/g in chloroform, respectively. Decreasing the polymer molecular weight has resulted in microparticles with a smaller average diameter 5.64 μm (±1.12) in F6 vs 6.83 μm (±1.18) in F1, as described above. As a result, F6 had a lower average drug loading (±SD) of 5.84% (±3.55) vs 8.25% (±3.73) in F1. This is because when a polymer of lower molecular weight is used, the inner oily phase has lower viscosity which offers less resistance to the shearing force during the emulsification process resulting in a smaller particle size. In addition, due to the lower viscosity of the oily phase, it becomes easier for the drug to escape to the external aqueous phase resulting in lower drug loading as seen in F6 (Xue et al., 2004; Jereswekin et al., 2007; Ynag et al., 2004; Gabor et al., 1999). Changing the polymer molecular weight also had a notable effect on the drug release profiles. FIG. 21d shows the cumulative release profiles of F1 and F6. Lowering the polymer molecular weight in F6 resulted in a stronger initial burst release and an overall faster release rate specially during the first 4 weeks. After 3 days, 23.7% (±5.6) and 51.4% (±9.48) of the encapsulated drug has been released from F1 and F6, respectively. This is due to the higher hydrophilicity of polymers with lower molecular weight resulting in faster degradation of the polymer and faster release (Han et al., 2016; Jereswekin et al., 2007). This trend was reversed after the 30-day mark, where F1 had a slightly faster release than F6, releasing 66.4% (±16.9) and 62.6% (±8.7) by day 42, respectively. The reason for this could be due to the precipitation of the high molecular weight polymer used in F1 at the bulk erosion phase, this polymer precipitation creates pores in the matrix which could be responsible for the faster release in the last two weeks compared to F6.

### In Vivo Pharmacokinetics Study

Based on the microparticles characterization data (particle size, drug loading, encapsulation efficiency) and in vitro release kinetics, formulation 1 was selected to further evaluate its in vivo release and pharmacokinetics in mice. This is because formulation 1 had acceptable formulation parameters compared to other formulations which was reflected in good drug loading and encapsulation efficiency values (±SD) of 8.25 (±3.73) and 90.7 (±41.1). In addition, to an appropriate particle size (±SD) of 6.83 μm (±1.18) making it injectable (Parke t al., 2019) and an in vitro release profile characterized by a small burst release and an overall slow release as shown in FIG. 21A. The in vivo plasma levels of ivacaftor following the intra-venous administration of 5 mg/kg solubilized ivacaftor and 50 mg/kg ivacaftor microparticles (formulation 1) to mice are shown in FIG. 22. The initial burst release of ivacaftor from the microparticles at 4 h (approx. 1 μg/mL) is approximately 100 times that of the average steady state plasma concentration (approx. 0.1 μg/mL) later in time. This difference between initial burst release plasma concentration and steady state plasma concentration is common after the sub cutaneous administration of microparticles and has been reported in Zhu et al. (2020). Sustained mice plasma levels of ivacaftor were observed up to 28 days following the SC administration of ivacaftor microparticle formulation 1 with concentrations ranging from 0.02-1.12 μg/mL. In contrast to the rapid elimination of ivacaftor following its intravenous administration to mice, where the last detectable plasma concentration was after only 1 day of administration. The area under the curve (AUC₀₋ₜ) estimates resulting from non-compartmental pharmacokinetic analysis (NCA) of the ivacaftor mice plasma data following the administration of 0.1 mg soluble ivacaftor and 1 mg ivacaftor microparticle formulation 1 has shown an approx. 6-fold increase in exposure reflected in AUC₀₋ₜ values of 71.6 and 12.3 (μg/mL*h) for ivacaftor microparticles and soluble ivacaftor, respectively. These results indicate that the SC administration of ivacaftor microparticles can eliminate the need of frequent administration of ivacaftor to treat CF by providing sustained plasma levels over a long period of time (28 days).

| | Dose amount (mg) | AUC (μg/ml*h) (NCA) |
|---|---|---|
| Ivacaftor IV solution | 0.1 | 12.3 |
| Ivacaftor SC microparticles | 1 | 71.6 |

### Conclusion

Ivacaftor (VX-770) is a member of a class of drugs used to treat CF called CFTR modulators. It is marketed by Vertex® pharmaceuticals since 2012 in an oral tablets form under the brand name Kalydeco®. To achieve therapeutic levels in CF patients, Kalydeco® is administered at a dose of 150 mg twice daily. Frequent daily administration is known to cause less compliance and adherence to treatment, specially for CF patients due to the multiple medications they administer. In this research we successfully provided a potential solution to this problem by encapsulating ivacaftor, for the first time, in long-acting PLGA-based microspheres using a single oil-in-water emulsion solvent evaporation method. By varying some of the formulation parameters (Drug:polymer ratio, surfactant concentration, emulsification speed, and polymer molecular weight) we developed an ivacaftor-loaded microparticle formulation (formulation 1) with optimum characteristics. The microparticle formulation had optimum particle size with an i.d (±SD) of 6.83 μm (±1.18) rendering it suitable for injection. The drug loading and encapsulation efficiency (±SD) of the microparticle formulation were 8.25% (±3.73), and 90.7% (±41.1). Good drug loading allows the injection of an appropriate amount of particles to achieve the target dose, in addition, 90% encapsulation efficiency means that almost all of the drug has been encapsulated in the formulation with minimal loss meaning that this formulation can be easily scaled up for entering human clinical trials without fear of being expensive due to drug loss. The in vitro release profile of the formulation showed the typical biphasic release pattern observed with PLGA based microparticles, with a small burst release and an overall slow cumulative release. When we injected the microparticle formulation SC into mice, ivacaftor had steady plasma levels that was detectable up to 28 days. When compared to solubilized ivacaftor injected intra-venously, the microparticle formulation increased the plasma exposure of ivacaftor and bioavailability 6 times reflected in AUC₀₋ₜ values of 12.3 and 71.6 (μg/ml*h) following the intravenous administration of 0.1 mg solubilized ivacaftor, and the subcutaneous administration of 1 mg ivacaftor microparticle formulation 1, respectively. These results suggest, for the first time, a potential clinically translatable, injectable ivacaftor-loaded PLGA based microparticle formulation that can eliminate the need for frequent twice daily administration of oral Kalydeco and thus improve patient compliance and adherence to the medication and the overall CF treatment outcomes.

### Example 8

Many polymer types have been investigated to develop formulations mediating sustained drug release with improved efficacy. PLGA possesses many desirable properties that make it an attractive candidate for use in such systems. In addition to its biocompatibility and low toxicity, it also offers predictable biodegradation kinetics. Due to the safety profile of PLGA, it has been approved by the USFDA as well as the European Medicines Agency for clinical use. It hydrolyzes into two monomers, lactic acid and glycolic acid, which are endogenous substances that can be utilized by the Krebs cycle, therefore, it is considered as biodegradable and biocompatible polymer. PLGA is also a good candidate for oral formulations because of its bioadhesive properties that allows binding to gastrointestinal mucosa, thereby extending the residence time of the encapsulated drug in the gastrointestinal tract leading to increased absorption period. However, oral delivery suffers the disadvantage of drug loss (e.g., first pass metabolism). Another advantage of using PLGA polymers is that they can be processed into a variety of shapes and sizes and are compatible with a wide variety of organic solvents. PLGA-based carriers such as microparticles can be readily loaded with distinct biomolecules or drugs and have been shown to be capable of differential release kinetics of drugs from the same formulation. Also, PLGA is highly tunable with respect to its molecular weight, lactide:glycolide ratios and functionalized end groups. Ciprofloxacin, an antibiotic, which is widely used for treatment of P. aeruginosa lung infections in CF patients, was encapsulated with high loading in PLGA nano-carriers. With these properties, PLGA has been used to deliver molecules with diverse physicochemical properties including small molecules, macromolecules, as well as hydrophilic and hydrophobic molecules/drugs.

### Materials and Methods

F7 (see details for F1A above) is a pellet formulation produced by a specific polymer (PLGA 75:25) with a specific viscosity/molecular weight (0.5-0.75 dl/g) with 4.5 g polymer amount and 1.5 g ivacaftor. Specific HME parameters (below) were used to produce a pellet with a specific thickness (average 0.7 mm) suitable for injecting SC using a G16 needle. For F8 different parameters were used (below) because the polymer has a different viscosity so to produce a pellet with same thickness the parameters were modified.

### Hot Melt Extruder (HME) Parameters

Hot melt extruder parameters were chosen to obtain a filament of thickness less than 1.19 mm (which is the thickness of the internal diameter of G16 needle) to be able to inject animals without the need for surgery. Other needles, e.g., G18, G17, G15, G14, G13 and the like, may be employed that are compatible with the thickness of the pellet.

For F7, following are the parameters for the formulation of PLGA pellets:
Current=0.3A; Voltage=2.1V; Extruding RPM=20; Extruding Temperature=180° C.; Conveyer belt speed=08
For F8, following are the parameters for the formulation of PLGA pellets:
Current=0.5A; Voltage=3.0V; Extruding RPM=30; Extruding Temperature=150° C.: Conveyer belt speed=08

### Experimental Design

Hot-melt extrusion (HME) was used to develop the Ivacaftor-loaded long-acting PLGA pellets.

| F# | Polymer | Amount of polymer | Ivacaftor |
|---|---|---|---|
| F7 | PLGA 75:25 | 4.5 g | 1.5 g |
| F8 | PLGA 50:50 | 4.5 g | 1.5 g |

To formulate a biodegradable pellet capable of sustained release of the drug, Ivacaftor, after injection, injectable pellets made from PLGA were fabricated using hot-melt extrusion (HME), in which pellets were loaded with Ivacaftor drug at a varying polymer:drug ratios and were tested for their ability to provide sustained release of the drug in in-vitro settings (PBS and 0.4% Tween-80 in PBS at 37° C.). Although PLGA microparticles have been a common means of drug delivery in preclinical studies, such a system is not appropriate in the context of a few drugs. To avoid the rapid loss of the drug during preparation and to minimize the burst release upon implantation, water-free fabrication methods of larger sized drug delivery platforms are desirable. The thermostable nature of Ivacaftor makes it a suitable candidate drug for loading into PLGA pellets using hot-melt extrusion (HME). Thus, hot-melt extrusion was used to create Ivacaftor-loaded PLGA pellets with different drug:polymer ratios.

### Results and Interpretation:

Ivacaftor loaded pellets were developed using PLGA 75:25 and a hot-melt extruder. The average thickness of the pellet was less than 1.19 mm and is suitable to be injected via G16 needle, e.g., no need for surgery to administer. The above synthesized PLGA pellets (F7) were cut into small pieces nearly weighing 10-20 mg and dissolved in 1 mL of acetonitrile. Drug loading analysis was done using Agilent HPLC and the dilution was done 100 fold using 50:50 ACN:water. Blank and drug-loaded pellets were produced with uniform average thickness and the corresponding data is shown below:

| | | DL (ug/mg) | EE | Yield |
|---|---|---|---|---|
| F# | Thickness (±SD) | (±SD) | % (±SD) | (%) (w/w) |
| F7 | 0.754 mm (±0.065) | 73.76 ± 15.74 | 22.2% | 52.9% |
| F8 | 0.80 mm ± 0.5 mm | | | |

### In Vitro Release Kinetics Study

Preparing release medium:

Release medium 1 (PBS, pH=7.4):
5 tablets of phosphate buffered saline were dissolved in 1 L nanopure water to make a phosphate buffer solution (PBS, pH=7.4).
   Release medium 2 (0.4% Tween 80 in PBS, pH=7.4)
5 tablets of phosphate buffered saline were dissolved in 1 L nanopure water to make a phosphate buffer solution (PBS, pH=7.4) then a total of 4 mL Polysorbate 80 was added to make a 0.4% Tween 80 solution in PBS.

### Release Study:

Ivacaftor pellets (F7-PLGA 75:25) were cut into 10-15 mg pieces equivalent to 0.9-1.1 mg of free Ivacaftor based on the drug loading. Pellets were submerged in 1 mL release medium (n=5 in PBS and n=5 in 0.4% Tween 80/PBS) and transferred to orbital shaking incubator (300 rpm, 37° C.). At each time point, 100 μL of the release medium was collected and analyzed by HPLC and replenished with fresh 100 μL release medium.

There is greater drug release with Tween-80/PBS as compared to PBS alone, and this may be attributed to the higher solubility of Ivacaftor in the Tween-80/PBS release medium.

Elexacaftor-Tezacaflor-Ivacaftor is a triple-combination cystic fibrosis transmembrane conductance regulator (CFTR) modulating therapy that contains 2 correctors and a potentiator of the CFTR channel. This drug combination provides potential therapy to many patients who had previously been excluded from CFTR modulation therapy due to the nature of their genetic mutations. This triple combination of drugs is given as a fixed-dose combination tablet of Elexacaftor 100 mg, Tezacaftor 50 mg, and Ivacaftor 75 mg co-packaged with ivacaftor 150 mg tablets. Adults and children over the age of 12 should administer 2 fixed-dose combination tablets each morning with a fat-containing meal. The evening dose should be separated by approximately 12 hours from morning administration and consists of 1 ivacaftor 150-mg tablet taken with a fat-containing meal or snack.

A triple combination (Elexacaftor-Tezacaftor-Ivacaftor) long acting PLGA pellets may avoid the daily/frequent administration of CFTR modulators.

### REFERENCES

Ahmed et al., Nat. Nanotechnol., 59:72 (2020).
Bahareh et al., J. Eng. Fiber Fabr., _:_ (2014).
Chen et al., J. Biomed. Mater. Res. A., 70:412 (2004).
Cheng et al., J. Control Rel., 5:203 (1998).
Clancy et al., Clin. Pharmacol. Ther., 95:592 (2014).
Condren & Bradshaw, J. Pediatr. Pharmacol. Ther., 18:8 (2013).
Conway et al., Thorax., 51:29 (1996).
Deeks, Drugs, 76:1191 (2016).
Ebeid et al., Nat. Nanotechnol., 13:72 (2018).
Fohner et al., Pharmacogenet. Genomics, 27:39 (2020).
Gaaz et al., Molecules, 20:22833 (2015).
Gabor et al., J. Microencapsul., 6:_ (1999).
Gross et al., Bioconjug. Chem., 31:2147 (2020).
Han et al., Front Pharmacol., 7:1 (2016).
Hedges, Starch, _:833 (2009).
Hoy, Drugs, 79:2001 (2019).
Ikpa et al., Int. J. Biochem. Cell Biol., 52:192 (2014).
Jaraswekin et al., J. Microencapsul., 24:117 (2007).
Joshi et al., AAPS J., 15:85 (2013).
Khaled et al., AAPS PharmSciTech. 11:859 (2010).
Lagreca et al., Prog Biomater., _:_ (2020).
Leelakanok et al., Journal of Pharmaceutical Sciences, 107:690 (2018).
Lima et al., Food Chem. Toxicol., 126:15 (2019).
Liu, Drug Delivery Systems Based on Polymer Blends: Synthesis, Characterization & Application. PhD Thesis presented to the faculty of Drexel University, USA. (2003).
Mao et al., Eur. J. Pharm. Biopharm., 6:214 (2008).
Naguib et al., Drug Deliv. Transl. Res., _:_ (2020).
Naguib et al., MS Thesis presented to the faculty of pharmacy Minia University, Egypt (2009).
Naguib et al., Mol. Pharm., 17:3643 (2020).
Nair & Laurencin, Prog. Polym. Sc., 22:762 (2007).
Park et al., J. Control Rel., _:_ (2020).
Park et al., J. Control Rel., 304:125 (2019).
Porsio et al., ACS Apl. Mater. Interfaces, 10:165 (2018).
Porsio et al., Int. J. Pharm., 582:119304 (2020).
Quarterman et al., Eur. J. Pharm. Biopharm., _:_ (2020).
Ridley et. al., J. Pediatr. Pharmacol. Ther., 25:192 (2020).
Rowe et al., Ann. Am. Thorac. Soc., 14:213 (2017).
Singh & Udupa, Pharm. Acta Helveitiae., _:165 (1997).
Türeli et. al., European Journal of Pharmaceutics and Biopharmaceutics, 117:363 (2017).
Wafa et al., Acta Biomater., 50:417 (2017)
Wafa et al., Nanomedicine Nanotechnology. Biol. Med., 21:102055 (2019).
Wafa et al., J. Pharmacol. Exp. Ther., 370:855 (2019).
Xue, Artif. Cells Blood Substit. Immobil. Biotechnol., 32:575 (2004).
Yang and Owusu-Ababio, Drug Dev. Ind. Pharm., 26:61 (2000).
Yang et al., Drug Deliv. J. Deliv. Target Ther. Agents, 8:93 (2001).
Zemanick et. al., Am. J. Respir. Crit. Care Med., 23Q:1522 (2021).
Zhang et al., J. Agric. Food Chem., 61:151 (2013).
Zhang et al., AAPS J., 12:263 (2010).
Zhu et al., Mol. Pharm., 17:3270 (2020).
Leelakanok et al., Journal of Pharmaceutical Sciences, 107:690 (2018).

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A composition comprising a carrier comprising a cyclodextrin or a non-ionic surfactant and one or more cystic fibrosis transmembrane conductance regulator (CFTR) modulators.

2. The composition of claim 1 wherein at least one of the CFTR modulators is a CFTR potentiator or a CFTR corrector or wherein the composition comprises at least one CFTR potentiator and at least one CFTR corrector.

3. The composition of claim 1 wherein the CFTR modulator comprises ivacaftor, GLPG2451, GLPG1837, QBW251, PTI-808, FDL176, FDL169, VX-770, PG-01, tetrahydrobenzothiophene, quercetin, genistein, rattlesnake phospholipase A2 or aminoarylthiazone or the CFTR corrector comprises lumacaftor, Corr-4a, VRT-325, tezacaftor, elexacaftor, cavosonstat, FDL169, VX-809, VX-152, VX-440, VX-445, or VX-659.

4-9. (canceled)

10. The composition of claim 1, wherein the carrier comprises the cyclodextrin which optionally comprises α-CD β-CD, γ-CD, HP-β-CD, M-β-CD, or HP-γ-CD, or a binary or a ternary mixture thereof, and optionally wherein the molar ratio between the CFTR modulator and the cyclodextrin is 3:1 to 1:10.

11-12. (canceled)

13. The composition of claim 1 wherein the carrier is the non-ionic surfactant which optionally comprises a polyol, glycol, glycerol ester or a sorbitan or ethoxylated modified polyol, polysorbate 80, polysorbate 20, ethanol, polyethylene glycol, propylene glycol, or a mixture of thereof.

14-17. (canceled)

18. The composition of claim 1 which is a tablet or a suspension of a powder of the one or more CFTR modulators in a pharmaceutically acceptable oral vehicle.

19-20. (canceled)

21. A method to prepare a composition comprising a cyclodextrin and one or more CFTR modulators, comprising; mixing one or more CFTR modulators and a cyclodextrin in a solution comprising water and an alcohol at ratios of 1:5 to 5:1; and drying the resulting mixture.

22-24. (canceled)

25. A method to inhibit or treat cystic fibrosis in a mammal, comprising: administering to a mammal in need thereof an effective amount of the composition of claim 1, and optionally further comprising administering at least one CFTR corrector or at least one CFTR potentiator.

26. The method of claim 25 wherein the mammal is a human.

27. The method of claim 25 wherein the composition is orally, intramuscularly, subcutaneously or intravenously administered.

28-38. (canceled)

39. The method of claim 25 28 wherein the composition comprises ivacaftor and at least one of lumacaftor, tezacaftor, or elexacaftor.

40-45. (canceled)

46. The method of claim 25 wherein the CFTR in the mammal comprises G551D, G178R, G551S, S549N, S549R, G970R, G1244E, S1251N, S1255P, or G1349D.

47. A composition comprising particles comprising one or more CFTR modulators and a diameter of about 1 μm to about 10 μm.

48. (canceled)

49. The composition of claim 47 which provides for sustained release of the one or more CFTR modulators.

50. The composition of claim 47 wherein the average diameter of the particles is about 2 μm to about 10 μm, about 3 μm to about 9 μm or about 4 μm to about 8 μm or have a thickness of about 0.5 mm to about 1.3 mm or about 0.6 mm to about 0.8 mm or about 0.5 mm to about 1.0 mm, or about 0.1 mm to about 0.5 mm.

51. The composition of claim 47 wherein the particles are biocompatible and biodegradable, formed of a polyester, formed of a natural polymer, or formed of lactic acid, glycolic acid, or combinations thereof.

52-54. (canceled)

55. The composition of claim 47 wherein the one or more CFTR modulators comprise ELX-02, PTC124, VX-809, VX-661, VX-445, VX-659, VX-440, PTI-801, ABBV-2222, ABBV-3221, FDL169, N91115, VX-770, VX-561, GLPG-1837, or PTI-428.

56-57. (canceled)

58. A method to prevent, inhibit or treat one or more symptoms of cystic fibrosis in a mammal, comprising administering to the mammal an effective amount of the composition of claim 47.

59. (canceled)

60. The method of claim 58 wherein the composition is injected, locally administered or systemically administered.

61-62. (canceled)

63. The method of claim 58 wherein the composition comprises particles formed of a polymer having a Mw of about 24,000 to about 38,000, wherein the one or more CFTR modulators are released from the particles over 2 to 6 weeks, wherein the particles are formed of lactic acid, glycolic acid, or combinations thereof or wherein the weight ratio of CFTR modulator to particle is about 1:5, 1:10, 1:15 or 1:20.

64-66. (canceled)
